(19) United States
(12) Patent Application Publication
(10) Pub. No.: US 2018/0094286 A1
(43) Pub. Date: Apr. 5, 2018

(54) PRODUCTION OF TERPENES AND TERPENOIDS

(71) Applicant: Technische Universitaet Graz, Graz (AT)

(72) Inventors: Thomas Vogl, Graz (AT); Anton Glieder, Gleisdorf (AT); Parayil Kumaran Ajikumar, Cambridge, MA (US)

(73) Assignee: Technische Universitaet Graz, Graz (AT)

(21) Appl. No.: 15/281,152

(22) Filed: Sep. 30, 2016

(51) Int. Cl. C12P 15/00; C12N 9/10; C12N 9/88

(52) U.S. Cl. CPC C12P 15/00; C12N 9/1085; C12Y 402/03017; C12Y 205/01001; C12N 9/88

(57) ABSTRACT

The present invention relates to a nucleic acid construct comprising a nucleic acid molecule encoding a protein involved in the biosynthesis of a terpenoid or a precursor thereof, wherein said nucleic acid molecule is operably linked to a derepressible promoter.

The present invention relates to a nucleic acid construct comprising a nucleic acid molecule encoding a protein involved in the biosynthesis of a terpenoid or a precursor thereof and uses of said construct in the biosynthesis of terpenoids or precursors thereof.

### REFERENCE TO A SEQUENCE LISTING SUBMITTED ELECTRONICALLY VIA EFS-WEB

The content of the electronically submitted sequence listing (Name: 47475_20160908_SEQ_LIST_ST25_txt.txt; Size: 36 kb; and Date of Creation: Sep. 30, 2016) is herein incorporated by reference in its entirety.

### BACKGROUND OF THE INVENTION

Terpenes and terpenoids are an important class of natural compounds widely used as dyes, flavors and pharmaceuticals. Both groups are derived from isoprene units, but in contrast to terpenes, terpenoids contain additional functional groups and consist not only of hydrocarbons. Many plant secondary metabolites such as the antimalarial drug artemisinin are terpenoids. Also steroids such as testosterone found in vertebrates are terpenoids. Especially for pharmaceutical applications, terpenoids are needed in large quantities and therefore scalable, economic production processes are required. Isolations from natural sources such as plants are limited by poor productivity and scalability.

Taxol and structurally related taxanes, for instance, are terpenoids naturally produced by yew trees (e.g. Taxus brevifolia). Taxol and Docetaxel are potent anticancer drugs, as they inhibit breakdown of microtubules thereby hampering the segregation of chromosomes and impairing mitotic cell division. However, Taxol is naturally only occurring in the bark of the Pacific yew tree (T. brevifolia) and two to four trees had to be cut down to allow treatment of a single patient. Various chemical synthesis routes have been reported requiring due to the complex structure containing 11 chiral centers at least 35 steps and a maximum yield of 0.4%. Nowadays Taxol is obtained from chemical synthesis, plant-cell cultures and still isolated from yew trees. Plant cell cultures are limited in their productivity and scalability whereas chemical synthesis is non-optimal in regards of yields and environmental considerations (requirement of large quantities of solvents and intricate protecting groups).

Recombinant production of complex natural products such as terpenoids can be achieved by metabolically engineered microorganisms. Thereby the natural enzymes e.g. of plant derived biosynthetic pathways are expressed in a heterologous host system such as Escherichia coli, Saccharomyces cerevisiae or Pichia pastoris. Using the latter host cells compounds including flavonoids, terpenoids such as artemisinic acid (a precursor of the antimalarial drug artemisinin), and carotenoids have successfully been produced.

Also the production of Taxol precursors, for instance, has been achieved in metabolically engineered microorganisms, most notably in E. coli and yeast. However, the full natural biosynthesis of Taxol requires 19 distinct enzymatic steps. Also the production of other terpenoids is highly complex requiring multiple enzymatic steps. So far most efforts of recombinant taxane production focused on taxadiene, the first dedicated precursor requiring two additional enzymatic steps from natural intermediates of the methylerythritol-phosphate (MEP) pathway or mevalonate (MVA) pathway. The MEP and MVA pathways produce the building blocks for terpenoid synthesis: isopentenyl pyrophosphate (IPP) and dimethylallyl pyrophosphate (DMAPP). The MVA pathway occurs in higher eukaryotes and some bacteria. The MEP pathway (also termed ‘non-mevalonate pathway’) is a complementary pathway occurring e.g. in bacteria and plant plastids. For taxadiene synthesis further two enzymatic steps catalyzed by geranylgeranyl pyrophosphate synthetase (GGPPS) and taxadiene synthase (TDS) are required. Ajikumar et al. (Science 330(2010):70-4) metabolically engineered E. coli to produce ˜1 g/l taxadiene by fine-tuning the expression levels of MEP pathway genes and GGPPS and TDS. In S. cerevisiae production of taxadiene at considerably lower yields has been demonstrated. In general diterpenoid production in yeasts gives rather low yields compared to multi gram scale production of sesquiterpenes such as artemisinic acid or nootkatone. This was explained by the high toxicity of diterpenes for yeast.

The success of recombinant taxadiene production paves the way for the production of more complex Taxol precursors. However, for full Taxol synthesis from taxadiene, 17 more enzymatic steps are required. About half of the follow up reactions from taxadiene are catalyzed by a cascade of cytochrome P450 monooxygenases (CYPs). These eukaryotic monooxygenases are difficult to express in E. coli as prokaryotes lack the respective electron transfer machinery and cytochrome P450 reductases (CPR). In addition CYPs and CPRs are membrane proteins localized in the endoplasmic reticulum, which is not present in E. coli.

P. pastoris has been shown to be a highly favorable platform for CYP and CPR expression, outperforming E. coli, Saccharomyces cerevisiae and Yarrowia lipolytica in a comparative study and may therefore be a valuable expression platform for Taxol production.

It is an object of the present invention to provide means allowing the production of terpenoids and/or precursors thereof in host cells, in particular in yeast cells.

### BRIEF DESCRIPTION OF THE INVENTION

The present invention relates to a nucleic acid construct comprising a nucleic acid molecule encoding a protein involved in the biosynthesis of a terpenoid or a precursor thereof, wherein said nucleic acid molecule is operably linked to a derepressible promoter.

In an embodiment of the present invention the protein involved in the biosynthesis of a terpenoid or a precursor thereof is selected from the group consisting of geranylgeranyl diphosphate synthases or taxadiene synthases.

In a further embodiment of the present invention the derepressed promoter is selected from the group consisting of CAT1 promoter, FDH1 promoter, FLD1 promoter, PEX5 promoter, DAK1 promoter, FGH1 promoter, GTH1 promoter, G1 promoter, G2 promoter, G3 promoter, G4 promoter, G5 promoter, G6 promoter, FMD promoter and a functional variant thereof. These promoters and their sequences are disclosed, for instance, in Vogl T et al. (ACS Synth. Biol. 5(2016):172-186) and Prielhofer R et al. (Microb Cell Fact 12(2013):5).

In an embodiment of the present invention, the derepressible promoter is operably linked with the geranylgeranyl pyrophosphate synthase gene.

In an embodiment of the present invention the promoter is an orthologous promoter.

In an embodiment of the present invention the derepressible promoter is linked to a second promoter forming a bidirectional promoter or a bidirectional derepressible promoter.

In a further embodiment of the present invention the second promoter is a constitutive, derepressed or inducible promoter.

In an embodiment of the present invention the constitutive promoter is selected from the group consisting of a GAP promoter, PGCW14 promoter, TEF1 promoter, TPI promoter, PGK1 promoter or a histone promoter.

In a further embodiment of the present invention the inducible promoter is selected from the group consisting of an AOX1 promoter or promoters which are regulated by the presence of a specific carbon source such as promoters of the methanol utilization (MUT) pathway, AOX2, DAS1, DAS2, FLD1, GTH1, PEX8 and PHO89/NSP.

In an embodiment of the present invention the bidirectional promoter comprises a combination of a GAP promoter, a CAT1 promoter, a PGCW14 promoter, a TEF1 promoter, a TPI promoter, a PGK1 promoter or a histone promoter, a promoter of the methanol utilization (MUT) pathway, a FDH1 promoter, a FLD1 promoter, a PEX5 promoter, a DAK1 promoter, a FGH1 promoter, a GTH1 promoter, a G1 promoter, a G2 promoter, a G3 promoter, a G4 promoter, a G5 promoter, a G6 promoter or a FMD promoter.

In an embodiment of the present invention the second promoter is operably linked to a second nucleic acid molecule encoding a second protein involved in the biosynthesis of a terpenoid or a precursor thereof.

In an embodiment of the present invention the CAT1 promoter is operably linked to a nucleic acid molecule encoding for a geranylgeranyl diphosphate synthase.

In an embodiment of the present invention the nucleic acid molecule encoding the protein involved in the biosynthesis of a terpenoid or a precursor thereof comprises a terminator sequence at its 3′ end.

Another aspect of the present invention relates to a vector comprising a nucleic acid construct according to the present invention.

Another aspect of the present invention relates to a host cell comprising a nucleic acid construct or a vector according to the present invention.

In an embodiment of the present invention the host cell is a yeast cell.

In an embodiment of the present invention said host cell is a methylotrophic yeast cell.

In an embodiment of the present invention the methylotrophic yeast cell is selected from the group of Pichia pastoris, Hansenula polymorpha (Ogataea polymorpha), Candida boidinii, Komagataella pastoris, Komagataella phaffii, Komagataella populi, Komagataella pseudopastoris, Komagataella ulmi and Komagataella sp. 11-1192.

Another aspect of the present invention relates to a method for producing a terpenoid or a precursor thereof comprising the step of cultivating a host cell according to the present invention.

### BRIEF DESCRIPTION OF THE FIGURES

FIG. 1 shows a nucleic acid construct according to the present invention wherein a bidirectional promoter (BDP) is inserted between GGPPS and TDS (see FIG. 1). The arrows indicate the orientation of the promoters of the BDP and the transcription direction of GGPPS and TDS.

FIG. 2 shows nucleotide sequences of bidirectional promoters.

FIG. 3 shows taxadiene (first dedicated precursor of taxol) production in P. pastoris using bidirectional promoters. The graph shows that there is a 50-fold difference in taxadiene yields depending on the promoter used. The indicated BDPs were cloned between the enzymes TDS and GGPPS and transformed into P. pastoris. The strains were cultivated in shake flasks with a dodecane overlay and induced with methanol as described in the example. Taxadiene yields were determined by GC-MS. Mean values and standard deviation of biological triplicates shown.

FIG. 4 shows the production of taxadiene under different cultivation conditions using a P. pastoris strain harboring TDS-pGAP|pCAT1-GGPPS. The use of a cultivation medium comprising 3% glycerol resulted in the production of up to 9.4 mg/l taxadiene. The strains were cultivated for 60 h on the glycerol concentrations indicated.

### DETAILED DESCRIPTION OF THE INVENTION

The present invention relates to a nucleic acid construct comprising a nucleic acid molecule encoding a protein involved in the biosynthesis of a terpenoid or a precursor thereof, wherein said nucleic acid molecule is operably linked to a derepressible promoter.

It turned surprisingly out that polypeptides and proteins involved in the biosynthesis of a terpenoid or a precursor thereof show high enzymatic activity if these polypeptides and proteins are expressed in a host cell using a derepressible promoter. In contrast thereto, the expression of these polypeptides and proteins using solely inducible or constitutive promoters operably linked to the respective nucleic acid molecules resulted in a significantly lower enzymatic activity, whereby this enzymatic activity is determined by measuring the production of the terpenoid or a precursor thereof.

“Nucleic acid construct”, as used herein, refers to any nucleic acid molecule such as cDNA, genomic DNA, synthetic DNA, semi synthetic DNA and RNA.

“A protein involved in the biosynthesis of a terpenoid or a precursor thereof”, as used herein, refers to proteins and polypeptides which are part of the biosynthetic pathways leading to terpenoids or precursors of the final compound. These proteins are either enzymatically active or influence directly the activity of enzymes involved in these pathways.

“Terpenoids”, as used herein, refers to a large and diverse class of organic molecules derived from five-carbon isoprenoid units assembled and modified in a variety of ways and classified in groups based on the number of isoprenoid units used in group members. The term “terpenoids” includes therefore also hemiterpenoids, monoterpenoids, sesquiterpenoids, diterpenoids, sesterterpenoids, triterpenoids, tetraterpenoids and polyterpenoids.

The term “terpenoid precursor” refers to any molecule that is used by organisms in the biosynthesis of terpenoids. Terpenoid precursor molecules can be any isoprenoid substrate molecule of terpene synthases such as peranylpyrophosphate, farnesylpyrophosphate or geranylgeranylpyrophosphate, and/or initial products made by terpene synthases such as amorphadiene, taxadiene, hopene, limonene (Degenhardt J et al. Phytochemistry 70(2009):1621-37).

“Operably linked”, as used herein, means that the promoter of the present invention is fused to nucleic acid molecule encoding a protein involved in the biosynthesis of a terpenoid or a precursor thereof to be able to regulate and influence the transcription of said nucleic acid molecule into RNA which thereafter is translated into the protein involved in the biosynthesis of a terpenoid or a precursor thereof.

As used herein, the term “promoter” refers to a nucleic acid sequence that is generally located upstream of a gene (i.e., towards the 5′ end of a gene) and is necessary to initiate and drive transcription of the gene. A promoter may permit proper activation or repression of a gene that it controls. A promoter includes a core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive, inducible or derepressible. The promoters of the present invention are preferably operable in yeast cells, in particular in methylotrophic yeast cells such as Pichia pastoris. These promoters are therefore preferably derived/obtained/isolated from yeast cells, in particular in methylotrophic yeast cells such as Pichia pastoris or are viral promoters which are functional in yeasts or synthetic promoters active in yeasts.

A “constitutive promoter” refers to one that is always active and/or constantly directs transcription of a gene above a basal level of transcription.

An “inducible promoter” is one which is capable of being induced by a molecule or a factor added to the cell or expressed in the cell. An inducible promoter may still produce a basal level of transcription in the absence of induction, but induction typically leads to significantly more production of the protein.

A “derepressible promoter”, as used herein, refers to a promoter that is substantially less active in prescence of a repressing compound. By changing the environment, repression is alleviated from the derepressible promoter and transcription rate increases. For instance, for some derepressible promoters glucose or glycerol can be used. Such promoters are repressed in the presence of glucose or glycerol and start expression once glucose or glycerol in the media is depleted.

According to a preferred embodiment of the present invention the protein involved in the biosynthesis of a terpenoid or a precursor thereof is selected from the group consisting of geranylgeranyl diphosphate synthases (GGPPS) and taxadiene synthases (TDS).

The protein involved in the biosynthesis of a terpenoid or a precursor thereof is particularly preferred geranylgeranyl diphosphate synthase (GGPPS).

According to a further preferred embodiment of the present invention the derepressible promoter is selected from the group consisting of CAT1 promoter, FDH1 promoter, FLD1 promoter, PEX5 promoter, DAK1 promoter, FGH1 promoter, GTH1 promoter, G1 promoter, G2 promoter, G3 promoter, G4 promoter, G5 promoter, G6 promoter, FMD promoter and a functional variant thereof, whereby a CAT1 promoter is particularly preferred.

A “functional variant” of a promoter, as used herein, refers to a promoter or a functional fragment thereof containing changes in relation to the wild-type promoter sequence which affect one or more nucleotides of the sequence. These nucleotides may be deleted, added and/or substituted, while maintaining at least substantially promoter function. The promoter function of functional promoter variants or fragments can be tested by operably linking a promoter variant or fragment to a nucleic acid molecule encoding a protein and evaluation the expression rate of the expressed protein or the transcription rate. Variant promoters can be produced, for example, by standard DNA mutagenesis techniques or by chemically synthesizing the variant promoter or a portion thereof.

“Functional variants” of promoters are at least 80%, preferably at least 85%, more preferably at least 90%, more preferably at least 95%, more preferably at least 97%, more preferably at least 98%, more preferably at least 99%, identical to the wild-type promoter sequence.

“Identical”, as used herein, refers to two or more sequences or subsequences that are the same or have a specified percentage of nucleotides that are the same, when compared and aligned for maximum correspondence, as measured using sequence comparison algorithms. It is particularly preferred to use BLAST and BLAST 2.0 algorithms (see e.g. Altschul et al. J. MoI. Biol. 215(1990): 403-410 and Altschul et al. Nucleic Acids Res. 25(1977): 3389-3402) using standard or default parameters. For amino acid sequences, the BLASTP program (see http://blast.ncbi.nlm.nih.gov/Blast.cgi) uses as defaults a wordlength (W) of 6, an expectation (E) of 10 and the BLOSUM62 scoring matrix (see Henikoff and Henikoff, Proc. Natl. Acad. Sci. USA 89(1989):10915) using Gap Costs Existance:11 Extension:1.

Functional variants of promoters include also “functional fragments” of promoters. The functional fragments of the promoters of the present invention retain at least substantially the promoter function of the entire promoter from which they are derived from. A functional fragment of a promoter may comprise at least 30%, preferably at least 40%, more preferably at least 50%, more preferably at least 60%, more preferably at least 65%, more preferably at least 70%, more preferably at least 75%, more preferably at least 80%, more preferably at least 85%, more preferably at least 90%, more preferably at least 95%, more preferably at least 97%, more preferably at least 98%, more preferably at least 99%, of the length of the entire promoter. A functional fragment of a promoter may comprise at least 100 consecutive bp, preferably at least 150 consecutive bp, more preferably at least 200 consecutive bp, more preferably at least 300 consecutive bp, more preferably at least 400 consecutive bp, more preferably at least 500 consecutive bp, of a wild type promoter.

The CAT1 promoter preferably comprises or consists of 100 to 500, 200 to 500, 300 to 500, 400 to 500 or 500 consecutive nucleotides of following nucleic acid sequence (see Vogl T et al., ACS Synth. Biol. 5(2016):172-186) (SEQ ID No. 1):

TAATCGAACTCCGAATGCGGTTCTCCTGTAACCTTAATTGTAGCATAGAT
CACTTAAATAAACTCATGGCCTGACATCTGTACACGTTCTTATTGGTCTT
TTAGCAATCTTGAAGTCTTTCTATTGTTCCGGTCGGCATTACCTAATAAA
TTCGAATCGAGATTGCTAGTACCTGATATCATATGAAGTAATCATCACAT
GCAAGTTCCATGATACCCTCTACTAATGGAATTGAACAAAGTTTAAGCTT
CTCGCACGAGACCGAATCCATACTATGCACCCCTCAAAGTTGGGATTAGT
CAGGAAAGCTGAGCAATTAACTTCCCTCGATTGGCCTGGACTTTTCGCTT
AGCCTGCCGCAATCGGTAAGTTTCATTATCCCAGCGGGGTGATAGCCTCT
GTTGCTCATCAGGCCAAAATCATATATAAGCTGTAGACCCAGCACTTCAA
TTACTTGAAATTCACCATAACACTTGCTCTAGTCAAGACTTACAATTAAA

The FDH1 promoter preferably comprises or consists of the following nucleic acid sequence (SEQ ID No. 2):

tagatggttatcttgaatggtatttgtaaggattgatctcgaaggttgta
tatagtcgtgccgtgcaagtggaggagaatgaaagaagatgtaagaattc
tggcccttgcacctgatcgcgaaggtggaaatggcagaaggatcagcctg
gacgaagcaaccagttccaactgctaagtaaagaagatgctagacgaagg
agacttcagaggtgaaaagtttgcaagaagagagctgcgggaaataaatt
ttcaatttaaggacttgagtgcgtccatattcgtgtacgtgtccaactgt
tttccattacctaagaaaaacataaagattaaaaagataaacccaatcgg
gaaactttagcgtgccgtttcggattccgaaaaacttttggagcgccaga
tgactatggaaagaggagtgtaccaaaatggcaagtcgggggctactcac
cggatagccaatacattctctaggaaccagggatgaatccaggtttttgt
tgtcacggtaggtcaagcattcacttcttaggaatatctcgttgaaagct
acttgaaatcccattgggtgcggaaccagcttctaattaaatagttcgat
gatgttctctaagtgggactctacggctcaaacttctacacagcatcatc
ttagtagtcccttcccaaaacaccattctaggtttcggaacgtaacgaaa
caatgttcctctcttcacattgggccgttactctagccttccgaagaacc
aataaaagggaccggctgaaacgggtgtggaaactcctgtccagtttatg
gcaaaggctacagaaatcccaatcttgtcgggatgttgctcctcccaaac
gccatattgtactgcagttggtgcgcattttagggaaaatttaccccaga
tgtcctgattttcgagggctacccccaactccctgtgcttatacttagtc
taattctattcagtgtgctgacctacacgtaatgatgtcgtaacccagtt
aaatggccgaaaaactatttaagtaagtttatttctcctccagatgagac
tctccttcttttctccgctagttatcaaactataaacctattttacctca
aatacctccaacatcacccacttaaaca

The FLD1 promoter preferably comprises or consists of the following nucleic acid sequence (SEQ ID No. 3):

tgtgaatatcaagaattgtatgaacaagcaaagttggagctttgagcgat
gtatttatatgagtagtgaaatcctgattgcgatcaggtaaggctctaaa
aatcgatgatggtcccgaattctttgataggctaaggacttcctcatcgg
gcagttcgaaggaagaaggggcatgagccctgcgaaaccatatgaggaag
ggagatagaagcagaagattatccttcgggagcaagtctttccagcccgc
atcttgtgattggatgatagttttaactaaggaaagagtgcgacatccgt
tgtgtagtaatcatgcatacgtctattattctctctagttacccaactct
gttatctcactaattcatggaatgccctccaggtagatactacaacgatt
caatagtactgcaacacacagatgagattagtttagtttcccataatgag
aattcagagtacaagaacaatctagtagccataagcaaggttcaccctct
cctgtttttatcctataggcggcatatccagatatatcgactacctcagc
tccgttggataactaccattagcaccgtgccagagattcctgca

The PEX5 promoter preferably comprises or consists of the following nucleic acid sequence (SEQ ID No. 4):

tccaaaccaaacggtctagcaaaaacgataactttaaagaacttttcaat
tggttttgtacactaccaccggtttactacctctgccttcggttcttctc
ctcacatttttcgcaactgggatagcgtagcctaaagtgtcacatgctcg
ctgctcacattccctacacaacagagattgtcagcagaggaaattgagct
ccaccattcaacacttgtggatttatgatagtctgtgctatcagctctct
tttttttgttgctgtagaatttaccgtgctagcaaccttttaaactttgt
ttagctctccttccctcttccattcatctgtttcggtccgatccgtctct
ggtcatctcctccgcattttttttttaccgttagcgataggggtcagatc
aattcaatcagttttggcaagggtatttaaaggtggcgaaatccccctcc
gtttgttgaacacatccaactattctcaacccaaccatctaactaatcgt
a

The DAK1 promoter preferably comprises or consists of the following nucleic acid sequence (SEQ ID No. 5):

tgtcatctgctgatgctgtgagggagaaagaagtaggggtgatacatggt
ttataggcaaagcatgtttgtttcagatcaaagattagcgtttcaaagtt
gtggaaaagtgaccatgcaacaatatgcaacacattcggattatctgata
agtttcaaagctactaagtaagcccgtttcaagtctccagaccgacatct
gccatccagtgattttcttagtcctgaaaaatacgatgtgtaaacataaa
ccacaaagatcggcctccgaggttgaacccttacgaaagagacatctggt
agcgccaatgccaaaaaaaaatcacaccagaaggacaattcccttccccc
ccagcccattaaagcttaccatttcctattccaatacgttccatagaggg
catcgctcggctcattttcgcgtgggtcatactagagcggctagctagtc
ggctgtttgagctctctaatcgaggggtaaggatgtctaatatgtcataa
tggctcactatataaagaacccgcttgctcaaccttcgactcctttcccg
atcctttgcttgttgcttcttcttttataacaggaaacaaaggaatttat
acactttaa

The FGH1 promoter preferably comprises or consists of the following nucleic acid sequence (SEQ ID No. 6):

atgtcatcaattactacttcaatcttcaaggtaacagctgaaatccaaag
ttttgggggaaagctagtcaaacttcaacacaagtccgatgagacgaaga
ctgacatggatgtgaacgtctaccttccagctcaattctttgccaatgga
gccaagggaaaatcattaccagttctactttatttgagtggtctgacttg
cactcccaacaatgcctcagagaaggcattttggcaaccatatgcaaata
agtacggttttgctgtggttttcccggatacttcacccagagggctcaac
atcgaaggagagcacgactcttatgattttggatccggtgccgggttcta
cgtggatgccactactgagaaatggaaggataattatagaatgtacagtt
atgttaactcggaattgctacccaaattgcaggctgacttcccaattcta
aactttgacaatatttcaatcacgggccactccatgggaggttacggagc
tttacagttattcttgagaaacccgggaaaattcaagtcggtttccgcat
tttctccaatctccaaccccactaaagccccatggggtgagaagtgcttc
tctggatacctgggacaggacaagtccacttggactcagtacgacccaac
cgaattgattggaaaataccaaggcccctcagattccagcattttgattc
acgttggaaagagtgattcgttctacttcaaggaccaccagctgctacct
gagaacttcttgaaggcttcagagaactctgtgttcaagggaaaagtgga
cttgaacttggtagatggctatgaccattcttactactttatctcttcat
tcacagacgttcatgctgctcaccatgcaaagtatttggggttaaactag

The G1 (GTH1) promoter preferably comprises or consists of the following nucleic acid sequence (SEQ ID No. 7):

ccccaaacatttgctccccctagtctccagggaaatgtaaaatatactgc
taatagaaaacagtaagacgctcagttgtcaggataattacgttcgactg
tagtaaaacaggaatctgtattgttagaaagaacgagagttttttacggc
gccgccatattgggccgtgtgaaaacagcttgaaaccccactactttcaa
aggttctgttgctatacacgaaccatgtttaaccaacctcgcttttgact
tgactgaagtcatcggttaacaatcaagtaccctagtctgtctgaatgct
cctttccatattcagtaggtgtttcttgcacttttgcatgcactgcggaa
gaattagccaatagcgcgtttcatatgcgcttttaccccctcttttgtca
agcgcaaaatgcctgtaagatttggtgggggtgtgagccgttagctgaag
tacaacaggctaattccctgaaaaaactgcagatagacttcaagatctca
gggattcccactatttggtattctgatatgtttttcctgatatgcatcaa
aactctaatctaaaacctgaatctccgctatttttttttttttttgatga
ccccgttttcgtgacaaattaatttccaacggggtcttgtccggataaga
gaattttgtttgattatccgttcggataaatggacgcctgctccatattt
ttccggttattaccccacctggaagtgcccagaattttccggggattacg
gataatacggtggtctggattaattaatacgccaagtcttacattttgtt
gcagtctcgtgcgagtatgtgcaataataaacaagatgagccaatttatt
ggattagttgcagcttgaccccgccatagctaggcatagccaagtgctat
gggtgttagatgatgcacttggatgcagtgagttttggagtataaaagat
ccttaaaattccaccctt

The G3 promoter preferably comprises or consists of the following nucleic acid sequence (SEQ ID No. 8):

cagcaatccagtaaccttttctgaatagcagagccttaactaaaataatg
gccagggtaaaaaattcgaaatttgacaccaaaaataaagacttgtcgtt
ataagtcttaacaaagtccgcaattttggagctaacggtggcggttgctg
ggatattcaataatggtagaatgttgctgcgggtatatgacagagcgtga
aacacactgaacaaggtaaatggaacaacagcaattgcaatatgggggag
gatagtcaagaacaaagcagcaatggcaaagtactgaatattctccaaag
ccaaaaggtccagtggtttcaacgacaaagtcttgttggtatagctttgg
aacaaaaggacaccgaaagactcgacagcgcccacaaatacagcgttgta
gaagaacgaattgattgctccagagcttctaatagtcagaagatacccca
aacctccgagcaacgttagcacatgacctaagaaccaggcgaagtgaaga
gtctggaataacgacacccagtcagtttttcctgagctcctggtgggatt
ggtagaagcatttgatttgcttggagtggttttatttgaagatggtgttg
aagccattgttgctaaagagtcggagttttgcttttagggtttgttaagc
aaaggaggaaaaactgcgccgtttgaagtcccaggtagtttcgcgtgtga
ggccagccagggaaagcttccttcggtacttttttttcttttgcaggttc
cggacggattaagcttcgggttatgaggggggcggtagccaattccggac
acaatattgcgtcgcagctagtcaccccgccataaatatacgcaggattg
aggtaataacatcgatagtcttagtaattaatacaattcagtggcgaatt
tggcaacatgacgtaaggcccactgttgtctataaaaggggatgaatttt
catgtttttgaggcctcccggacaatttattgaactcaa

The G4 promoter preferably comprises or consists of the following nucleic acid sequence (SEQ ID No. 9):

tggactgttcaatttgaagtcgatgctgacgatgtcaagagagatgctca
attatatttgtcatttgctggttacactggaaacgctacttttgttggcg
gaaactctaccagtttggccgtccatgtaaacgatgtcgttctgggccgt
gaccgtttcaacacgaacataaccaatgacaaatccacttacaggtctag
ttcatatggaggcaattggtaccttacttctttggatgtcccaagtgggg
ctttaacgtctggtactaacaatgtctcgtttgtcactacaaactccgag
gtaaataaaggattcttgtgggattctctcaagtttgtttggaagttgta
acaggtttataagcatatcgtgcgcttgtccacaattgaatcatttattg
ttgcgagatacatgaacaaagtgtgaactgggacccattactacaattcc
cacgcaaccgttgtttcaaagcccatattttttgacaattgtttcgttac
acccccagtttgatgtacatcgcttgcaatgatgtgtgtcccggagtatt
ttccatattcagcttgaattcgtatactcaaccaatatctgggggtatac
ttttatgtaacctatacaaatcaactatactatttcacctttcgaccatc
atctcccatcttgttaagttttgcttcctatatccctgaccctgacatca
cccatgattccgctcaacggttctcctctacatcgtccctcttttggaga
gggtgttcagtttgacattcaaattaccccccgccatcacgcgcaaccga
gaccgcacccccgaattttcacaaattaccccacaccctatactccacca
ctatgagggttattagaactgatcacgtataaataccaccgcaagttccc
aagggatcgtgttcttcttctccaattgcaatcatatttctgactctttc
tagttcagattaattcctttacacttgcttttttcccttacctttatcc

The G6 promoter preferably comprises or consists of the following nucleic acid sequence (SEQ ID No. 10):

ccagaccagcagtttaactacgcaaatccacaggaatttctacatcacaa
taccaatggtaataccacgacgtcaaggaatggaaacgacgacttggagg
aagacttcgtcaacctcttgcggagtacccgaggctaagacaataagaag
aaaaaaaaagaaaagcggtgggggagggattattaaataaggattatgta
accccagggtaccgttctatacatatttaaggattatttaggacaatcga
tgaaatcggcatcaaactggatgggagtatagtgtccggataatcggata
aatcatcttgcgaggagccgcttggttggttggtgagaggagtgaaatat
gtgtctcctcacccaagaatcgcgatatcagcaccctgtgggggacacta
ttggcctccctcccaaaccttcgatgtggtagtgctttattatattgatt
acattgattacatagctaaaccctgcctggttgcaagttgagctccgaat
tccaatattagtaaaatgcctgcaagataacctcggtatggcgtccgacc
ccgcttaattattttaactcctttccaacgaggacttcgtaatttttgat
tagggagttgagaaacggggggtcttgatacctcctcgatttcagatccc
accccctctcagtcccaagtgggacccccctcggccgtgaaatgcgcgca
ctttagtttttttcgcatgtaaacgccggtgtccgtcaattaaaagtcgc
agactagggtgaactttaccatttttgtcgcactccgtctcctcggaata
ggggtgtagtaattctgcagtagtgcaatttttaccccgccaaggggggg
cgaaaagagacgacctcatcacgcattctccagtcgctctctacgcctac
agcaccgacgtagttaactttctcccatatataaagcaattgccattccc
ctgaaaactttaacctctgctttttcttgatttttccttgcccaaagaaa
ag

| | Gene identifier (P. | |
|---|---|---|
| Promoter | pastoris GS115 strain) | Genbank Acc. No. |
| G7 | PAS_chr1-4_0570 | NC_012963.1 |
| G8 | PAS_chr1-3_0165 | NC_012963.1 |

The FMD promoter preferably comprises or consists of the following nucleic acid sequence (SEQ ID No. 11):

aatgtatctaaacgcaaactccgagctggaaaaatgttaccggcgatgcg
cggacaatttagaggcggcgatcaagaaacacctgctgggcgagcagtct
ggagcacagtcttcgatgggcccgagatcccaccgcgttcctgggtaccg
ggacgtgaggcagcgcgacatccatcaaatataccaggcgccaaccgagt
ctctcggaaaacagcttctggatatcttccgctggcggcgcaacgacgaa
taatagtccctggaggtgacggaatatatatgtgtggagggtaaatctga
cagggtgtagcaaaggtaatattttcctaaaacatgcaatcggctgcccc
gcAacgggaaaaagaatgactttggcactcttcaccagagtggggtgtcc
cgctcgtgtgtgcaaataggctcccactggtcaccccggattttgcagaa
aaacagcaagttccggggtgtctcactggtgtccgccaataagaggagcc
ggcaggcacggagtctacatcaagctgtctccgatacactcgactaccat
ccgggtctctcagagaggggaatggcactataaataccgcctccttgcgc
tctctgccttcatcaatcaaatc

The promoter comprised in the nucleic acid construct of the present invention can be an orthologous promoter.

The promoters used in the construction of the nucleic acid construct of the present invention can be of the same

“Orthologous promoter”, as defined herein, is a promoter derived from another organism, preferably from another yeast strain or species. Such promoters are derived from the same precursor promoter and have similar biological and/or biochemical characteristics.

According to a particularly preferred embodiment of the present invention the derepressible promoter is linked to a second promoter forming a bidirectional promoter.

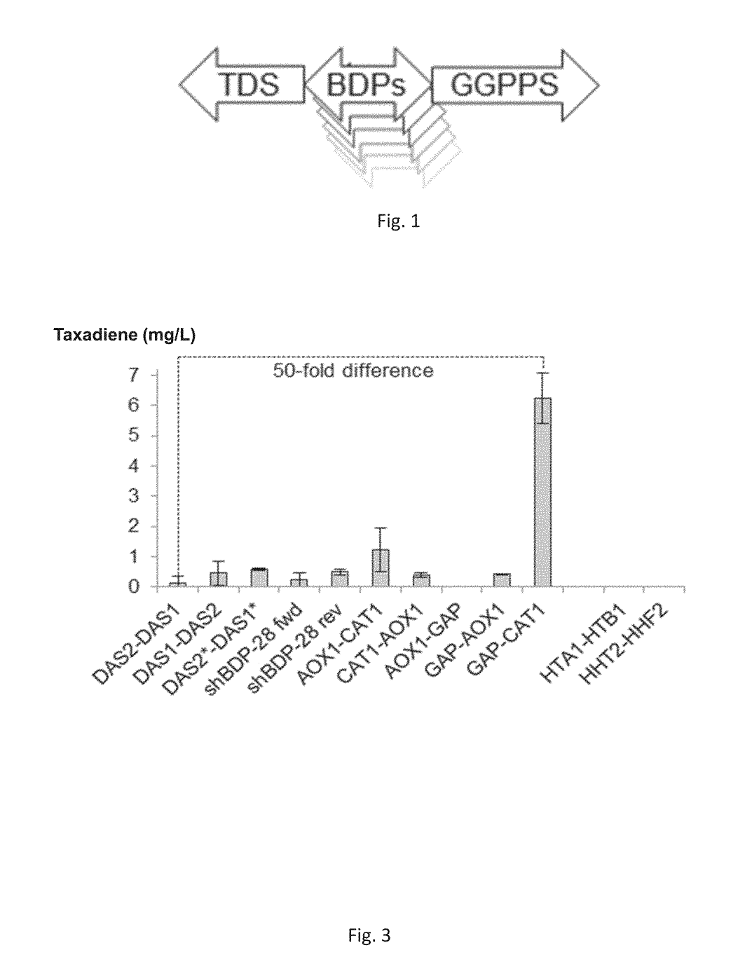

Bidirectional promoters are able of directing transcription in both the forward and reverse orientations. A bidirectional promoter can direct the transcription of two transcripts placed in either orientation (i.e., downstream or upstream) of the promoter simultaneously (e.g., the “sense” and “antisense” strands of a gene). In other words, a bidirectional promoter can direct transcription from either strand of the promoter region. The use of bidirectional promoters enables co-expression of two genes by placing them in opposing orientations and placing a bidirectional promoter in between them (see FIG. 1 and EP 2 862 933). The two promoters within the bidirectional promoter may be separated by a linker comprising or consisting of 1 to 500, preferably 1 to 300, more preferably 1 to 200, more preferably 1 to 100, more preferably 1 to 50, nucleotides.

The second promoter of the bidirectional promoter can be a constitutive, derepressible or inducible promoter. Hence, the bidirectional promoter of the present invention comprises a derepressible promoter and constitutive or inducible promoter in inverse orientation.

The constitutive promoter is preferably selected from the group consisting of a GAP promoter, PGCW14 promoter, TEF1 promoter, TPI promoter, PGK1 promoter or a histone promoter (see e.g. Vogl T et al. (ACS Synth. Biol. 5(2016):172-186)).

The inducible promoter is preferably selected from the group consisting of promoters of the methanol utilization (MUT) pathway, preferably selected from the group consisting of AOX1 promoter, AOX2 promoter, DAS1 promoter, DAS2 promoter, FLD1 promoter, GTH1 promoter, PEX8 promoter or PHO89/NSP promoter (see e.g. Vogl T et al. (ACS Synth. Biol. 5(2016):172-186)).

According to a preferred embodiment of the present invention the bidirectional promoter comprises a combination of the aforementioned promoters preferably a combination of two promoters selected from the group consisting of a GAP promoter, a CAT1 promoter, a PGCW14 promoter, a TEF1 promoter, a TPI promoter, a PGK1 promoter, a histone promoter, a promoter of the methanol utilization (MUT) pathway, preferably a AOX1 promoter, a AOX2 promoter, a DAS1 promoter, a DAS2 promoter, a FLD1 promoter, a GTH1 promoter, a PEX8 promoter or a PHO89/NSP promoter, a FDH1 promoter, a FLD1 promoter, a PEX5 promoter, a DAK1 promoter, a FGH1 promoter, a GTH1 promoter, a G1 promoter, a G2 promoter, a G3 promoter, a G4 promoter, a G5 promoter or a G6 promoter.

Particularly preferred is a bidirectional promoter comprising a CAT1 promoter in combination with a GAP promoter or a promoter of the methanol utilization (MUT) pathway, preferably a AOX1 promoter, or two CAT1 promoters without any other promoter.

The order of the various promoters within the bidirectional can be any whereby particularly preferred are GAP-CAT1 and AOX1-CAT1 promoters.

According to a further preferred embodiment of the present invention the second promoter is operably linked to a second nucleic acid molecule encoding a second protein involved in the biosynthesis of a terpenoid or a precursor thereof.

Proteins involved in the biosynthesis of a terpenoids or precursors thereof and nucleic acid molecules encoding said proteins are known in the art. These proteins are also involved in the biosynthesis of terpenoid precursor molecules (i.e. any isoprenoid substrate molecule) and include terpene synthases such as peranylpyrophosphate, farnesylpyrophosphate or geranylgeranylpyrophosphate, and/or initial products made by terpene synthases such as amorphadiene, taxadiene, hopene, limonene (see e.g. Degenhardt J et al. Phytochemistry 70(2009):1621-37).

According to a particular preferred embodiment of the present invention the CAT1 promoter is operably linked to a nucleic acid molecule encoding for a geranylgeranyl diphosphate synthase.

It turned out that a CAT1 promoter controlling the expression of geranylgeranyl diphosphate synthase allows obtaining high product yields.

In order to stop transcription of a nucleic acid molecule into mRNA and to release the nascent transcript it is advantageous to provide terminator sequence at the 3′ end of a coding region to be transcribed. Hence, the nucleic acid molecule of the present invention encoding the protein involved in the biosynthesis of a terpenoid or a precursor thereof comprises preferably a terminator sequence at its 3′ end.

Another aspect of the present invention relates to a vector comprising a nucleic acid construct according to the present invention.

The vector of the present invention can be used to deliver the nucleic acid construct of the invention into a host cell, for instance.

A further aspect of the present invention relates to a host cell comprising a nucleic acid construct or a vector according to the present invention.

The nucleic acid construct and the vector of the present invention can be part of a host cell. The host cell can harbor these molecules for cloning purposes and/or for expressing the coding regions/genes present in these nucleic acid molecules. Depending on the host cell the nucleic acid construct and the vector of the present invention may comprise additional elements like antibiotic resistance genes and genetic markers.

The host cell of the present invention is preferably a yeast cell, preferably a methylotrophic yeast cell.

According to a preferred embodiment of the present invention the methylotrophic yeast cell is selected from the group of Pichia pastoris, Hansenula polymorpha (Ogataea polymorpha), Candida boidinii, Komagataella pastoris, Komagataella phaffii, Komagataella populi, Komagataella pseudopastoris, Komagataella ulmi and Komagataella sp. 11-1192.

Another aspect of the present invention relates to a method for producing a terpenoid or a precursor thereof comprising the step of cultivating a host cell according to the present invention.

The host cell of the present invention comprises a nucleic acid construct comprising a nucleic acid molecule encoding a protein involved in the biosynthesis of a terpenoid or a precursor thereof which is operably linked to a derepressible promoter. In order to express the aforementioned protein derepressible conditions have to be used. These conditions can vary and depend on the derepressible promoter to be used.

The present invention is further illustrated in the following examples, however, without being restricted thereto.

### Example

Materials and Methods

Plasmids

Codon optimized GGPPS (geranylgeranyl diphosphate synthase) and TDS (taxadiene synthase) genes were used for taxadiene production in P. pastoris. The genes were synthesized as double stranded DNA fragments with suitable overhangs for Gibson assembly.

TABLE A
Entry vectors

| | | SEQ ID |
|---|---|---|
| Name | Sequence | No. |
| p_aox1_syn-swai- | cagatcgggaacactgaaaaatacacagttattattcatttaa | 12 |
| das1tt-3prime-gib | atgacccttgtgactgacactttgggagtc | |
| aox1tt-5prime- | caggcaaatggcattctgacatcctcttgagcggccgcacggg | 13 |
| noti-das1tt- | aagtctttacagttttagttaggag | |
| 5prime-gib | | |
| intarg4-sbfi- | gtagatatttataccattctgcgagaaggtcccctgcagggac | 14 |
| das1tt-3prime-gib | ccttgtgactgacactttgggagtc | |
| gblock- | atgtttgatttcaatgagtacatgaagtctaaggccgttgcag | 15 |
| ggpps_opttv- | ttgatgcagctctggataaggctatcccactggagtacccaga | |
| aox1tt-gib | aaagatccacgaatctatgagatactctttgcttgcaggtgga | |
| | aagagagttagacctgctctttgcattgctgcttgtgagttgg | |
| | ttggaggttctcaagaccttgctatgccaactgcttgcgccat | |
| | ggagatgattcacactatgtctttgattcatgatgatttgcct | |
| | tgcatggataacgacgacttccgtcgtggtaagcctaccaacc | |
| | acaaggttttcggtgaggacaccgctgttttggccggtgacgc | |
| | tctgttatctttcgctttcgaacacattgccgtcgcaacctct | |
| | aagaccgtgccttcagacagaacccttagagttatttcagagc | |
| | tgggtaagaccattggttctcaaggattggtcggaggacaagt | |
| | tgttgatattacttctgaaggtgacgcaaacgttgacctgaag | |
| | actttggaatggattcacatccacaaaactgccgtcttattgg | |
| | agtgttctgtcgtctctggaggaatcttgggaggagctaccga | |
| | ggacgagattgctagaattagaagatacgctcgttgcgttgga | |
| | ttgttattccaggttgttgacgatatccttgacgtcactaagt | |
| | cctccgaagaattgggtaaaactgctggtaaagaccttcttac | |
| | tgacaaggcaacctaccctaagttgatgggtctggaaaaagct | |
| | aaggagttcgcagctgagttggcaactcgtgctaaggaggaat | |
| | tgtcatcttttgatcaaatcaaggccgctccattgttgggatt | |
| | ggcagactatatcgcctttagacaaaactgatcaagaggatgt | |
| | cagaatgccatttgcctg | |
| tds-bmri-stuffer- | cttggaagtaccagtagaagaggacatagcacccagtggcgcg | 16 |
| gib | ccgacctctgttgcctctttgttggac | |
| ggpps-bmri- | cttagacttcatgtactcattgaaatcaaacatcagtcccagt | 17 |
| stuffer-gib | gagctcttaagctggaagagccaatctcttgaaag | |
| gblock-tds_opttv- | atgtcctcttctactggtacttccaaggttgtctctgaaactt | 18 |
| part1 | cctctaccatcgttgacgacatcccaagattgtcagccaacta | |
| | ccacggtgacttgtggcaccacaatgttatccagactttggaa | |
| | actcctttcagagaatcttccacttatcaggagagagctgacg | |
| | agctggtcgtcaagatcaaggacatgttcaacgctctgggtga | |
| | cggagacatctccccttctgcatacgatactgcttgggtggcc | |
| | cgtcttgccactatttcttccgacggttctgaaaagcctagat | |
| | tccctcaggctcttaattgggtcttcaataaccaattgcaaga | |
| | tggttcctggggaattgaatcccacttctctctttgtgacaga | |
| | cttttgaacactaccaattctgttatcgcactgtctgtgtgga | |
| | aaaccggtcattcccaggtccaacaaggtgctgagttcattgc | |
| | tgagaacttgagacttcttaacgaggaagacgagctttcccct | |
| | gattttcagatcattttcccagctttgcttcaaaaagctaaag | |
| | cattgggtattaacttgccttacgacttgcctttcattaagta | |
| | tttgtcaaccaccagagaagctcgtttaaccgacgtctccgct | |
| | gcagccgataacattccagcaaacatgttgaacgcccttgagg | |
| | gtttggaggaagttattgactggaacaagattatgagattcca | |
| | gtccaaggacggttctttcctttcttctccagcctctaccgcc | |
| | tgcgttttgatgaacactggagatgagaagtgttttacttttc | |
| | tgaacaacttgttggataaatttggtggttgcgttccatgtat | |
| | gtattcaatcgacctgttggagagattatcattggtggataac | |
| | atcgaacacttgggaatcggtcgtcacttcaagcaagaaatta | |
| | agggagctttggactatgtctacagacactggtctgagagagg | |
| | tattggttggggtcgtgattctttagtccctgacctgaacacc | |
| | actgctttgggtttgagaactcttagaatgcacggttacaatg | |
| | tttcttctgacgttttgaacaacttcaaggatgaaaacggtag | |
| | atttttctcctctgccggtcaaactcatgtcgagctgagatct | |
| | gttgtcaacttgttccgtgcttctgatttggcattcccagatg | |
| | aaagagctatggacgacgctagaaagtttg | |
| gblock-tds_opttv- | aagagctatggacgacgctagaaagtttgcagagccttacttg | 19 |
| part2-das1tt-gib | agagaagctctggccactaagatttctactaacactaaacttt | |
| | tcaaggagatcgagtacgttgtcgaatacccttggcacatgtc | |
| | tattccacgtcttgaagctagatcttacatcgattcttacgat | |
| | gacaactacgtttggcagcgtaagactttatacagaatgccat | |
| | cactttcaaactcaaagtgcttggaattggctaaactggactt | |
| | caacattgttcagtccttgcatcaggaggagttgaagttgttg | |
| | actagatggtggaaggaatcaggtatggccgatattaacttca | |
| | ccagacaccgtgttgctgaggtttacttctcctccgcaacctt | |
| | tgagccagagtattctgctactagaatcgctttcactaaaatt | |
| | ggttgcttacaagtcttgttcgatgacatggctgatatcttcg | |
| | ctactcttgacgagcttaagtctttcactgagggagttaagcg | |
| | ttgggacacttccttgttacacgaaattccagaatgtatgcag | |
| | acttgtttcaaagtctggttcaagttgatggaggaggttaata | |
| | acgatgttgttaaggtgcaaggtagagatatgttggctcacat | |
| | tcgtaagccttgggagttatacttcaactgttatgttcaagag | |
| | agagagtggcttgaggctggttacattccaacttttgaggaat | |
| | acttgaagacttacgctatctcagtcggtttgggtccttgcac | |
| | tttacaacctatcctgttgatgggtgagttagtcaaggacgac | |
| | gttgttgaaaaagttcactatccttctaacatgttcgaattgg | |
| | tgtctttgtcttggagattgactaacgacactaagacctacca | |
| | agcagagaaggctcgtggacaacaagcctctggtattgcttgt | |
| | tacatgaaagacaaccctggtgctaccgaggaagacgctatta | |
| | agcacatttgtagagttgtcgaccgtgctcttaaggaagcatc | |
| | atttgaatacttcaagccatccaacgatattccaatgggttgt | |
| | aagtctttcattttcaacttaagactgtgcgttcaaattttct | |
| | ataagttcattgacggttacggtatcgcaaacgaagagattaa | |
| | agattacattcgtaaggtctacattgacccaattcaagtctaa | |
| | acgggaagtctttacagttttagttaggag | |
| seqggpps_tvopt- | tctaactctctttccacctgcaag | 20 |
| 118..141rev | | |
| seqtds_opttv- | cagctctctcctgataagtggaag | 21 |
| 146..169rev | | |
| seqtds_opttv- | gatgaacactggagatgagaagtg | 22 |
| 783..806fwd | | |

The GGPPS and TDS genes were cloned in opposite orientation to insert bidirectional promoters (BDPs) in between them (see FIG. 1).

To facilitate cloning at first an intermediate vector providing two different transcription terminators (TAOX1 and TDAS1) in opposite orientation separated by a NotI restriction site was generated. If two genes (such as GGPPS and TDS) should be co-expressed, this vector can be used for insertion. Two different cloning vectors were prepared: pPpT4_S-DAS1TT-NotI-AOX1TT and pPpT4mutZeoMlyI-intArg4-DAS1TT-NotI-AOX1TT. The former is based on the pPpT4_S vector reported by Näätsaari et al. (PLoS One 7(2012):e39720): following NotI and SwaI digestion and purification of the backbone a PCR product of the TDAS1 bearing overhangs to the vector (primers: P_AOX1_Syn-SwaI-DAS1TT-3prime-Gib and AOX1TT-5prime-NotI-DAS1TT-5prime-Gib) was cloned by Gibson assembly (Gibson D G et al. Nat Methods 6(2009):343-5). The latter vector contained in addition a sequence to target specific genomic integration (intArg4) and a mutated MlyI site in the Zeocin resistance gene (silent mutation). This vector was generated by digesting the pPpT4mutZeoMlyI-intArg4-bidi-dTOM-eGFP-BmrIstuffer vector (see US 2015/0011407) with SbfI and NotI and inserting a PCR product containing the respective overhangs (primers: intARG4-SbfI-DAS1TT-3prime-Gib and AOX1TT-5prime-NotI-DAS1TT-5prime-Gib) by Gibson assembly.

An entry vector containing the GGPPS and TDS genes separated by a stuffer/placeholder fragment was generated. This vector for taxadiene coexpression was generated by using P. pastoris codon optimized GGPPS and TDS genes. The genes were provided as synthetic double stranded fragments (gBlocks by Integrated DNA Technologies) with overhangs for Gibson assembly (gBlock-GGPPS_optTV-AOX1TT-Gib, gBlock-TDS_optTV-Part1 and gBlock-TDS_optTV-Part2-DAS1TT-Gib). A stuffer fragment with complementary overhangs was amplified using primers TDS-BmrI-stuffer-Gib and GGPPS-BmrI-stuffer-Gib. The four fragments were mixed in equimolar ratios with the NotI digested pPpT4mutZeoMlyI-intArg4-DAS1TT-NotI-AOX1TT backbone and joined by Gibson assembly. This vector was named pPpT4mutZeoMlyI-intArg4-DAS1TT-AOX1TT-TDS_optTV-GGPPS_optTV-BmrIstuffer.

Finally the stuffer fragment was cut out by BmrI digestion and the BDPs cloned in by Gibson assembly. The primers used for amplification are provided in Table B.

TABLE B

| | | SEQ ID |
|---|---|---|
| Name | Sequence | No. |
| TDS-pDAS2-Gib | cttggaagtaccagtagaagaggacatttttgatgtttgatagtt | 23 |
| | tgataagagtgaac | |
| GGPPS-pDAS1- | gacttcatgtactcattgaaatcaaacatTTTGTTCGATTATTCT | 24 |
| Gib | CCAGATAAAATCAAC | |
| TDS-pDAS1-Gib | cttggaagtaccagtagaagaggacatTTTGTTCGATTATTCTCC | 25 |
| | AGATAAAATCAAC | |
| GGPPS-pDAS2- | gacttcatgtactcattgaaatcaaacatttttgatgtttgatag | 26 |
| Gib | tttgataagagtg | |
| TDS-HHT2-Gib | cttggaagtaccagtagaagaggacatTTTTACTACGATAGACAC | 27 |
| | AAGAAGAAGCAG | |
| GGPPS-HHF2- | gacttcatgtactcattgaaatcaaacatATTTATTGATTATTTG | 28 |
| Gib | TTTATGGGTGAGTC | |
| TDS-HHF2-Gib | cttggaagtaccagtagaagaggacatATTTATTGATTATTTGTT | 29 |
| | TATGGGTGAGTC | |
| GGPPS-HHT2- | gacttcatgtactcattgaaatcaaacatTTTTACTACGATAGAC | 30 |
| Gib | ACAAGAAGAAGCAG | |
| TDS-AOX1-Gib | cttggaagtaccagtagaagaggacatCGTTTCGAATAATTAGTT | 31 |
| | GTTTTTTGATC | |
| GGPPS-CAT1- | gacttcatgtactcattgaaatcaaacatTTTAATTGTAAGTCTT | 32 |
| Gib | GACTAGAGCAAGTG | |
| TDS-CAT1-Gib | cttggaagtaccagtagaagaggacatTTTAATTGTAAGTCTTGA | 33 |
| | CTAGAGCAAGTG | |
| GGPPS-AOX1- | gacttcatgtactcattgaaatcaaacatCGTTTCGAATAATTAG | 34 |
| Gib | TTGTTTTTTGATC | |
| TDS-GAP-Gib | cttggaagtaccagtagaagaggacatTGTGTTTTGATAGTTGTT | 35 |
| | CAATTGATTG | |
| GGPPS-GAP-Gib | gacttcatgtactcattgaaatcaaacatTGTGTTTTGATAGTTG | 36 |
| | TTCAATTGATTG | |
| pGAP-pCAT1- | gacgaggacaccaagacatttctacaaaaaTAATCGAACTCCGAA | 37 |
| Gib | TGCGGTTCTC | |
| TDS-HTA1 | cttggaagtaccagtagaagaggacatTGTTGTAGTTTTAATATA | 38 |
| | GTTTGAGTATG | |
| GGPPS-HTB1 | gacttcatgtactcattgaaatcaaacatTTTGATTTGTTTAGGT | 39 |
| | AACTTGAACTGGATG | |

The primer combinations for the amplification of the promoters are listed in Table C.

TABLE C

| Bidirectional | | |
|---|---|---|
| promoter | Primer 1 | Primer 2 |
| DAS2-DAS1 | TDS-pDAS2-Gib | GGPPS-pDAS1-Gib |
| DAS1-DAS2 | TDS-pDAS1-Gib | GGPPS-pDAS2-Gib |
| DAS2-d8-DAS1-d2d5 | TDS-pDAS2-Gib | GGPPS-pDAS1-Gib |
| shBDP-28 fwd | TDS-HHT2-Gib | GGPPS-HHF2-Gib |
| shBDP-28 rev | TDS-HHF2-Gib | GGPPS-HHT2-Gib |
| AOX1-CAT1 | TDS-AOX1-Gib | GGPPS-CAT1-Gib |
| CAT1-AOX1 | TDS-CAT1-Gib | GGPPS-AOX1-Gib |
| AOX1-GAP | TDS-AOX1-Gib | GGPPS-GAP-Gib |
| GAP-AOX1 | TDS-GAP-Gib | GGPPS-AOX1-Gib |
| GAP-CAT1 | TDS-GAP-Gib | GGPPS-CAT1-Gib |
| CAT1-GAP | TDS-CAT1-Gib | GGPPS-GAP-Gib |
| HTA1-HTB1 | TDS-HTA1 | GGPPS-HTB1 |
| HHT2-HHF2 | TDS-HHT2-Gib | GGPPS-HHF2-Gib |

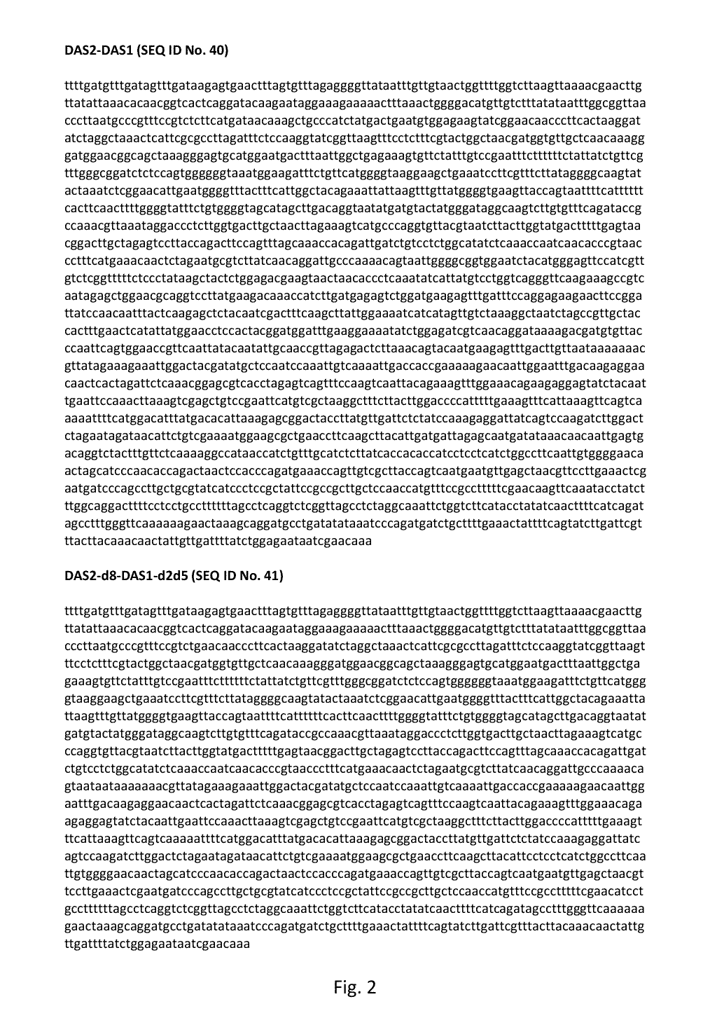

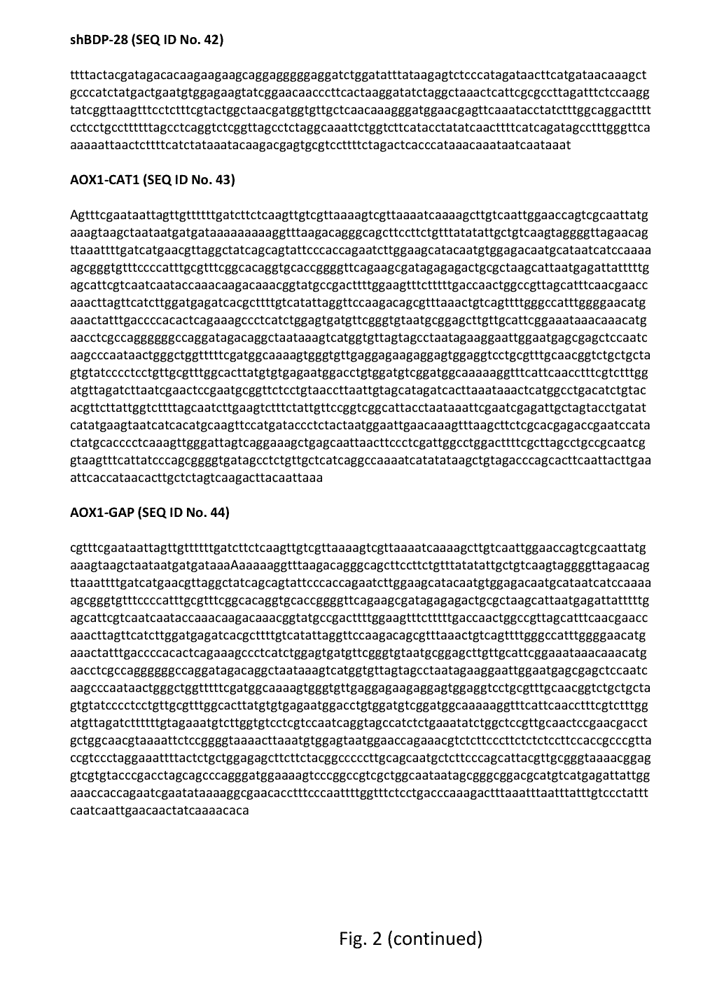

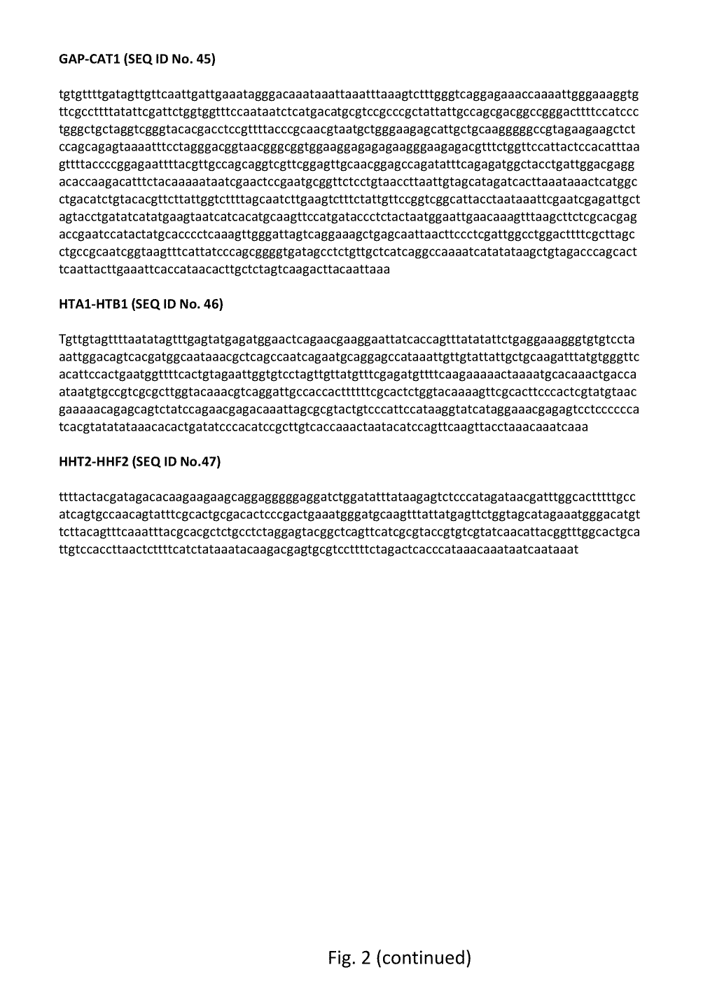

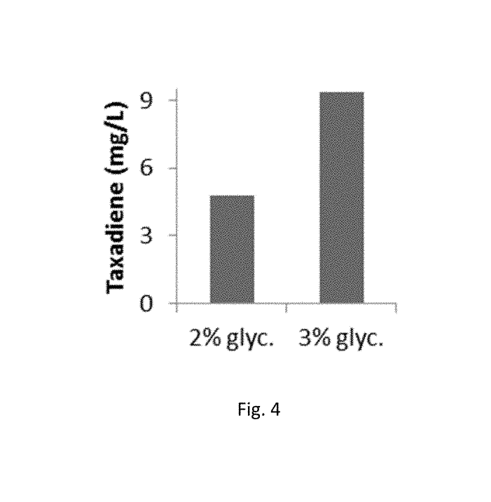

The nucleotide sequences of the bidirectional promoters (BDPs) obtained with the primers of Table B and used herein are depicted in FIG. 2.

Strains, Cultivation Conditions and Measurements

Pichia pastoris strain CBS7435 was used as host for transformation. Transformations of P. pastoris cells were performed with SwaI linearized plasmids following the condensed protocol by Lin-Cereghino et al. (Biotechniques 38(2005):44, 46, 48).

Taxadiene producing strains were cultivated in shake flasks in 50 ml buffered yeast peptone glycerol media (BYPG; 1% glycerol, 20 g/l peptone, 10 g/l yeast extract, 200 mM potassium phosphate buffer pH 6). A dodecane overlay of 10% of the volume (e.g. 5 ml) was added when the cultivation was started. In case methanol induction was performed, only 25 ml BYPG media were used and grown for 60 h, subsequently 25 ml BYPM2 media were added (1% (v/v) methanol). Methanol to 0.5% (v/v) was again added after 12, 24, 48 h and the shake flasks harvested after 72 h. For methanol induction, the dodecane overlay was added after growth on glycerol for 60 h together with the BMM2 addition. Selected strains were also cultivated on 2% and 3% BYPG media and harvested after 60 h.

The dodecane overlay was harvested by centrifugation at 3220 g for 25 min at 4° C. and analyzed by mass spectrometry for taxadiene contents (using a calibration curve based on peak areas comparison to a taxadiene standard curve).

Results

Diterpenoids are GGPP (geranylgeranyl diphosphate) derivatives. GGPP is produced by geranylgeranyl diphosphate synthase (GGPPS). The diterpenoid, taxadiene, is generated from mevalonate pathway products by two enzymatic steps: GGPPS and taxadiene synthase (TDS). The taxadiene production can be transcriptionally influenced by using differently regulated promoters (see FIG. 3), whereby bidirectional promoters (BDPs) have been using exemplarily in this example. The promoters featured similarly high expression levels, but combinations of different regulatory profiles on each side (constitutive, inducible and derepressed/derepressible activity). The yields obtained from P. pastoris strains transformed with plasmids bearing these BDPs spanned a 50-fold range.

P. pastoris strains expressing only TDS and GGPPS from a BDP reached yields comparable to a heavily engineered S. cerevisiae strain (6.2 mg/l mg/L vs. 8.7 mg/l; Engels B et al. Metab Eng 10(2008):201-6). Even in shake flasks the yields could be further improved by adapting the cultivation conditions, reaching 9.4 mg/l (FIG. 3).

This shows that the regulation of the expression of GGPPS is a key factor for high yields. Inducible or constitutive regulation suggested in literature resulted in 5- to 50-fold lower yields than derepressed regulation (activation when a repressing carbon source is depleted). Constitutive expression of the GGPPS appeared even lethal resulting in no taxadiene production at all.

These results suggest that host cells like P. pastoris alongside the flux optimization/transcriptional fine-tuning strategies outlined here, are a production platform for terpenoids such as Taxol precursors. Here, the methylotrophic yeast Pichia pastoris was used for controlled, balanced expression of terpenoid pathway genes, exemplified by the production of a diterpene, the Taxol precursor taxadiene. Unexpectedly, by transformation of a single plasmid into P. pastoris, higher taxadiene yields than in a highly engineered comparable S. cerevisiae strain (Engels B et al.) were obtained. Surprisingly, expression of GGPPS under derepressed conditions turned out to be a key factor for product high yields.

1. A nucleic acid construct comprising a nucleic acid molecule encoding a protein involved in the biosynthesis of a terpenoid or a precursor thereof, wherein said nucleic acid molecule is operably linked to a derepressible promoter.

2. The nucleic acid construct according to claim 1, wherein the protein involved in the biosynthesis of a terpenoid or a precursor thereof is selected from the group consisting of geranylgeranyl diphosphate synthases or taxadiene synthases.

3. The nucleic acid construct according to claim 1, wherein the derpressible promoter is selected from the group consisting of CAT1 promoter, FDH1 promoter, FLD1 promoter, PEX5 promoter, DAK1 promoter, FGH1 promoter, GTH1 promoter, G1 promoter, G2 promoter, G3 promoter, G4 promoter, G5 promoter, G6 promoter, FMD promoter and a functional variant thereof.

4. The nucleic acid construct according to claim 1, wherein the promoter is an orthologous promoter.

5. The nucleic acid construct according to claim 1, wherein the derepressible promoter is linked to a second promoter forming a bidirectional promoter or a bidirectional derepressible promoter.

6. The nucleic acid construct according to claim 5, wherein the second promoter is a constitutive, derepressible or inducible promoter.

7. The nucleic acid construct according to claim 6, wherein the constitutive promoter is selected from the group consisting of a GAP promoter, PGCW14 promoter, TEF1 promoter, TPI promoter, PGK1 promoter or a histone promoter.

8. The nucleic acid construct according to claim 6, wherein the inducible promoter is selected from the group consisting of a AOX1 promoter, promoters of the methanol utilization (MUT) pathway, AOX2, DAS1, DAS2, FLD1, GTH1, PEX8 and PHO89/NSP.

9. The nucleic acid construct according to claim 5, wherein the bidirectional promoter comprises a combination of a GAP promoter, a CAT1 promoter, a PGCW14 promoter, a TEF1 promoter, a TPI promoter, a PGK1 promoter or a histone promoter, a promoter of the methanol utilization (MUT) pathway, a FDH1 promoter, a FLD1 promoter, a PEX5 promoter, a DAK1 promoter, a FGH1 promoter, a GTH1 promoter, a G1 promoter, a G2 promoter, a G3 promoter, a G4 promoter, a G5 promoter, a G6 promoter or a FMD promoter.

10. The nucleic acid construct according to claim 9, wherein the promoter of the methanol utilization (MUT) pathway is selected from the group consisting of an AOX1 promoter, an AOX2 promoter, a DAS1 promoter, a DAS2 promoter, a FLD1 promoter, a GTH1 promoter, a PEX8 promoter or a PHO89/NSP promoter.

11. The nucleic acid construct according to claim 5, wherein the second promoter is operably linked to a second nucleic acid molecule encoding a second protein involved in the biosynthesis of a terpenoid or a precursor thereof.

12. The nucleic acid construct according to claim 3, wherein the derepressible promoter is operably linked to a nucleic acid molecule encoding for a geranylgeranyl diphosphate synthase.

13. The nucleic acid construct according to claim 3, wherein the CAT1 promoter is operably linked to a nucleic acid molecule encoding for a geranylgeranyl diphosphate synthase.

14. The nucleic acid construct according to claim 1, wherein the nucleic acid molecule encoding the protein involved in the biosynthesis of a terpenoid or a precursor thereof comprises a terminator sequence at its 3′ end.

15. A vector comprising a nucleic acid construct according to claim 1.

16. A host cell comprising a nucleic acid construct according to claim 1.

17. The host cell according to claim 16, wherein said host cell is a yeast cell.

18. The host cell according to claim 16, wherein said host cell is a methylotrophic yeast cell.

19. The host cell according to claim 16, wherein the methylotrophic yeast cell is selected from the group of Pichia pastoris, Hansenula polymorpha, Candida boidinii, Komagataella pastoris, Komagataella phaffii, Komagataella populi, Komagataella pseudopastoris, Komagataella ulmi and Komagataella sp. 11-1192.

20. A method for producing a terpenoid or a precursor thereof comprising the step of cultivating a host cell according to claim 16.


SEQUENCE LISTING (47 sequences)

SEQ ID NO: 1 - DNA, 500 bp, Artificial Sequence
  CAT1 promoter
taatcgaactccgaatgcggttctcctgtaaccttaattgtagcatagatcacttaaataaactcatggcctgacatctgtacacgttcttattggtcttttagcaatcttgaagtctttctattgttccggtcggcattacctaataaattcgaatcgagattgctagtacctgatatcatatgaagtaatcatcacatgcaagttccatgataccctctactaatggaattgaacaaagtttaagcttctcgcacgagaccgaatccatactatgcacccctcaaagttgggattagtcaggaaagctgagcaattaacttccctcgattggcctggacttttcgcttagcctgccgcaatcggtaagtttcattatcccagcggggtgatagcctctgttgctcatcaggccaaaatcatatataagctgtagacccagcacttcaattacttgaaattcaccataacacttgctctagtcaagacttacaattaaa

SEQ ID NO: 2 - DNA, 1128 bp, Artificial Sequence
  FDH1 promoter
tagatggttatcttgaatggtatttgtaaggattgatctcgaaggttgtatatagtcgtgccgtgcaagtggaggagaatgaaagaagatgtaagaattctggcccttgcacctgatcgcgaaggtggaaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaaca

SEQ ID NO: 3 - DNA, 594 bp, Artificial Sequence
  FLD1 promoter
tgtgaatatcaagaattgtatgaacaagcaaagttggagctttgagcgatgtatttatatgagtagtgaaatcctgattgcgatcaggtaaggctctaaaaatcgatgatggtcccgaattctttgataggctaaggacttcctcatcgggcagttcgaaggaagaaggggcatgagccctgcgaaaccatatgaggaagggagatagaagcagaagattatccttcgggagcaagtctttccagcccgcatcttgtgattggatgatagttttaactaaggaaagagtgcgacatccgttgtgtagtaatcatgcatacgtctattattctctctagttacccaactctgttatctcactaattcatggaatgccctccaggtagatactacaacgattcaatagtactgcaacacacagatgagattagtttagtttcccataatgagaattcagagtacaagaacaatctagtagccataagcaaggttcaccctctcctgtttttatcctataggcggcatatccagatatatcgactacctcagctccgttggataactaccattagcaccgtgccagagattcctgca

SEQ ID NO: 4 - DNA, 501 bp, Artificial Sequence
  PEX5 promoter
tccaaaccaaacggtctagcaaaaacgataactttaaagaacttttcaattggttttgtacactaccaccggtttactacctctgccttcggttcttctcctcacatttttcgcaactgggatagcgtagcctaaagtgtcacatgctcgctgctcacattccctacacaacagagattgtcagcagaggaaattgagctccaccattcaacacttgtggatttatgatagtctgtgctatcagctctcttttttttgttgctgtagaatttaccgtgctagcaaccttttaaactttgtttagctctccttccctcttccattcatctgtttcggtccgatccgtctctggtcatctcctccgcattttttttttaccgttagcgataggggtcagatcaattcaatcagttttggcaagggtatttaaaggtggcgaaatccccctccgtttgttgaacacatccaactattctcaacccaaccatctaactaatcgta

SEQ ID NO: 5 - DNA, 609 bp, Artificial Sequence
  DAK1 promoter
tgtcatctgctgatgctgtgagggagaaagaagtaggggtgatacatggtttataggcaaagcatgtttgtttcagatcaaagattagcgtttcaaagttgtggaaaagtgaccatgcaacaatatgcaacacattcggattatctgataagtttcaaagctactaagtaagcccgtttcaagtctccagaccgacatctgccatccagtgattttcttagtcctgaaaaatacgatgtgtaaacataaaccacaaagatcggcctccgaggttgaacccttacgaaagagacatctggtagcgccaatgccaaaaaaaaatcacaccagaaggacaattcccttcccccccagcccattaaagcttaccatttcctattccaatacgttccatagagggcatcgctcggctcattttcgcgtgggtcatactagagcggctagctagtcggctgtttgagctctctaatcgaggggtaaggatgtctaatatgtcataatggctcactatataaagaacccgcttgctcaaccttcgactcctttcccgatcctttgcttgttgcttcttcttttataacaggaaacaaaggaatttatacactttaa

SEQ ID NO: 6 - DNA, 900 bp, Artificial Sequence
  FGH1 promoter
atgtcatcaattactacttcaatcttcaaggtaacagctgaaatccaaagttttgggggaaagctagtcaaacttcaacacaagtccgatgagacgaagactgacatggatgtgaacgtctaccttccagctcaattctttgccaatggagccaagggaaaatcattaccagttctactttatttgagtggtctgacttgcactcccaacaatgcctcagagaaggcattttggcaaccatatgcaaataagtacggttttgctgtggttttcccggatacttcacccagagggctcaacatcgaaggagagcacgactcttatgattttggatccggtgccgggttctacgtggatgccactactgagaaatggaaggataattatagaatgtacagttatgttaactcggaattgctacccaaattgcaggctgacttcccaattctaaactttgacaatatttcaatcacgggccactccatgggaggttacggagctttacagttattcttgagaaacccgggaaaattcaagtcggtttccgcattttctccaatctccaaccccactaaagccccatggggtgagaagtgcttctctggatacctgggacaggacaagtccacttggactcagtacgacccaaccgaattgattggaaaataccaaggcccctcagattccagcattttgattcacgttggaaagagtgattcgttctacttcaaggaccaccagctgctacctgagaacttcttgaaggcttcagagaactctgtgttcaagggaaaagtggacttgaacttggtagatggctatgaccattcttactactttatctcttcattcacagacgttcatgctgctcaccatgcaaagtatttggggttaaactag

SEQ ID NO: 7 - DNA, 968 bp, Artificial Sequence
  G1 promoter
ccccaaacatttgctccccctagtctccagggaaatgtaaaatatactgctaatagaaaacagtaagacgctcagttgtcaggataattacgttcgactgtagtaaaacaggaatctgtattgttagaaagaacgagagttttttacggcgccgccatattgggccgtgtgaaaacagcttgaaaccccactactttcaaaggttctgttgctatacacgaaccatgtttaaccaacctcgcttttgacttgactgaagtcatcggttaacaatcaagtaccctagtctgtctgaatgctcctttccatattcagtaggtgtttcttgcacttttgcatgcactgcggaagaattagccaatagcgcgtttcatatgcgcttttaccccctcttttgtcaagcgcaaaatgcctgtaagatttggtgggggtgtgagccgttagctgaagtacaacaggctaattccctgaaaaaactgcagatagacttcaagatctcagggattcccactatttggtattctgatatgtttttcctgatatgcatcaaaactctaatctaaaacctgaatctccgctatttttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 8 - DNA, 989 bp, Artificial Sequence
  G3 promoter
cagcaatccagtaaccttttctgaatagcagagccttaactaaaataatggccagggtaaaaaattcgaaatttgacaccaaaaataaagacttgtcgttataagtcttaacaaagtccgcaattttggagctaacggtggcggttgctgggatattcaataatggtagaatgttgctgcgggtatatgacagagcgtgaaacacactgaacaaggtaaatggaacaacagcaattgcaatatgggggaggatagtcaagaacaaagcagcaatggcaaagtactgaatattctccaaagccaaaaggtccagtggtttcaacgacaaagtcttgttggtatagctttggaacaaaaggacaccgaaagactcgacagcgcccacaaatacagcgttgtagaagaacgaattgattgctccagagcttctaatagtcagaagataccccaaacctccgagcaacgttagcacatgacctaagaaccaggcgaagtgaagagtctggaataacgacacccagtcagtttttcctgagctcctggtgggattggtagaagcatttgatttgcttggagtggttttatttgaagatggtgttgaagccattgttgctaaagagtcggagttttgcttttagggtttgttaagcaaaggaggaaaaactgcgccgtttgaagtcccaggtagtttcgcgtgtgaggccagccagggaaagcttccttcggtacttttttttcttttgcaggttccggacggattaagcttcgggttatgaggggggcggtagccaattccggacacaatattgcgtcgcagctagtcaccccgccataaatatacgcaggattgaggtaataacatcgatagtcttagtaattaatacaattcagtggcgaatttggcaacatgacgtaaggcccactgttgtctataaaaggggatgaattttcatgtttttgaggcctcccggacaatttattgaactcaa

SEQ ID NO: 9 - DNA, 1000 bp, Artificial Sequence
  G4 promoter
tggactgttcaatttgaagtcgatgctgacgatgtcaagagagatgctcaattatatttgtcatttgctggttacactggaaacgctacttttgttggcggaaactctaccagtttggccgtccatgtaaacgatgtcgttctgggccgtgaccgtttcaacacgaacataaccaatgacaaatccacttacaggtctagttcatatggaggcaattggtaccttacttctttggatgtcccaagtggggctttaacgtctggtactaacaatgtctcgtttgtcactacaaactccgaggtaaataaaggattcttgtgggattctctcaagtttgtttggaagttgtaacaggtttataagcatatcgtgcgcttgtccacaattgaatcatttattgttgcgagatacatgaacaaagtgtgaactgggacccattactacaattcccacgcaaccgttgtttcaaagcccatattttttgacaattgtttcgttacacccccagtttgatgtacatcgcttgcaatgatgtgtgtcccggagtattttccatattcagcttgaattcgtatactcaaccaatatctgggggtatacttttatgtaacctatacaaatcaactatactatttcacctttcgaccaatcatctcccatcttgttaagttttgcttcctatatccctgaccctgacatcacccatgattccgctcaacggttctcctctacatcgtccctcttttggagagggtgttcagtttgacattcaaattaccccccgccatcacgcgcaaccgagaccgcacccccgaattttcacaaattaccccacaccctatactccaccactatgagggttattagaactgatcacgtataaataccaccgcaagttcccaagggatcgtgttcttcttctccaattgcaatcatatttctgactctttctagttcagattaattcctttacacttgcttttttcccttacctttatcc

SEQ ID NO: 10 - DNA, 1002 bp, Artificial Sequence
  G6 promoter
ccagaccagcagtttaactacgcaaatccacaggaatttctacatcacaataccaatggtaataccacgacgtcaaggaatggaaacgacgacttggaggaagacttcgtcaacctcttgcggagtacccgaggctaagacaataagaagaaaaaaaaagaaaagcggtgggggagggattattaaataaggattatgtaaccccagggtaccgttctatacatatttaaggattatttaggacaatcgatgaaatcggcatcaaactggatgggagtatagtgtccggataatcggataaatcatcttgcgaggagccgcttggttggttggtgagaggagtgaaatatgtgtctcctcacccaagaatcgcgatatcagcaccctgtgggggacactattggcctccctcccaaaccttcgatgtggtagtgctttattatattgattacattgattacatagctaaaccctgcctggttgcaagttgagctccgaattccaatattagtaaaatgcctgcaagataacctcggtatggcgtccgaccccgcttaattattttaactcctttccaacgaggacttcgtaatttttgattagggagttgagaaacggggggtcttgatacctcctcgatttcagatcccaccccctctcagtcccaagtgggacccccctcggccgtgaaatgcgcgcactttagtttttttcgcatgtaaacgccggtgtccgtcaattaaaagtcgcagactagggtgaactttaccatttttgtcgcactccgtctcctcggaataggggtgtagtaattctgcagtagtgcaatttttaccccgccaagggggggcgaaaagagacgacctcatcacgcattctccagtcgctctctacgcctacagcaccgacgtagttaactttctcccatatataaagcaattgccattcccctgaaaactttaacctctgctttttcttgatttttccttgcccaaagaaaag

SEQ ID NO: 11 - DNA, 623 bp, Artificial Sequence
  FMD promoter
aatgtatctaaacgcaaactccgagctggaaaaatgttaccggcgatgcgcggacaatttagaggcggcgatcaagaaacacctgctgggcgagcagtctggagcacagtcttcgatgggcccgagatcccaccgcgttcctgggtaccgggacgtgaggcagcgcgacatccatcaaatataccaggcgccaaccgagtctctcggaaaacagcttctggatatcttccgctggcggcgcaacgacgaataatagtccctggaggtgacggaatatatatgtgtggagggtaaatctgacagggtgtagcaaaggtaatattttcctaaaacatgcaatcggctgccccgcaacgggaaaaagaatgactttggcactcttcaccagagtggggtgtcccgctcgtgtgtgcaaataggctcccactggtcaccccggattttgcagaaaaacagcaagttccggggtgtctcactggtgtccgccaataagaggagccggcaggcacggagtctacatcaagctgtctccgatacactcgactaccatccgggtctctcagagaggggaatggcactataaataccgcctccttgcgctctctgccttcatcaatcaaatc

SEQ ID NO: 12 - DNA, 73 bp, Artificial Sequence
  Primer
cagatcgggaacactgaaaaatacacagttattattcatttaaatgacccttgtgactgacactttgggagtc

SEQ ID NO: 13 - DNA, 68 bp, Artificial Sequence
  Primer
caggcaaatggcattctgacatcctcttgagcggccgcacgggaagtctttacagttttagttaggag

SEQ ID NO: 14 - DNA, 68 bp, Artificial Sequence
  Primer
gtagatatttataccattctgcgagaaggtcccctgcagggacccttgtgactgacactttgggagtc

SEQ ID NO: 15 - DNA, 921 bp, Artificial Sequence
  Artificial Sequence
atgtttgatttcaatgagtacatgaagtctaaggccgttgcagttgatgcagctctggataaggctatcccactggagtacccagaaaagatccacgaatctatgagatactctttgcttgcaggtggaaagagagttagacctgctctttgcattgctgcttgtgagttggttggaggttctcaagaccttgctatgccaactgcttgcgccatggagatgattcacactatgtctttgattcatgatgatttgccttgcatggataacgacgacttccgtcgtggtaagcctaccaaccacaaggttttcggtgaggacaccgctgttttggccggtgacgctctgttatctttcgctttcgaacacattgccgtcgcaacctctaagaccgtgccttcagacagaacccttagagttatttcagagctgggtaagaccattggttctcaaggattggtcggaggacaagttgttgatattacttctgaaggtgacgcaaacgttgacctgaagactttggaatggattcacatccacaaaactgccgtcttattggagtgttctgtcgtctctggaggaatcttgggaggagctaccgaggacgagattgctagaattagaagatacgctcgttgcgttggattgttattccaggttgttgacgatatccttgacgtcactaagtcctccgaagaattgggtaaaactgctggtaaagaccttcttactgacaaggcaacctaccctaagttgatgggtctggaaaaagctaaggagttcgcagctgagttggcaactcgtgctaaggaggaattgtcatcttttgatcaaatcaaggccgctccattgttgggattggcagactatatcgcctttagacaaaactgatcaagaggatgtcagaatgccatttgcctg

SEQ ID NO: 16 - DNA, 70 bp, Artificial Sequence
  Primer
cttggaagtaccagtagaagaggacatagcacccagtggcgcgccgacctctgttgcctctttgttggac

SEQ ID NO: 17 - DNA, 78 bp, Artificial Sequence
  Primer
cttagacttcatgtactcattgaaatcaaacatcagtcccagtgagctcttaagctggaagagccaatctcttgaaag

SEQ ID NO: 18 - DNA, 1234 bp, Artificial Sequence
  Artificial Sequence
atgtcctcttctactggtacttccaaggttgtctctgaaacttcctctaccatcgttgacgacatcccaagattgtcagccaactaccacggtgacttgtggcaccacaatgttatccagactttggaaactcctttcagagaatcttccacttatcaggagagagctgacgagctggtcgtcaagatcaaggacatgttcaacgctctgggtgacggagacatctccccttctgcatacgatactgcttgggtggcccgtcttgccactatttcttccgacggttctgaaaagcctagattccctcaggctcttaattgggtcttcaataaccaattgcaagatggttcctggggaattgaatcccacttctctctttgtgacagacttttgaacactaccaattctgttatcgcactgtctgtgtggaaaaccggtcattcccaggtccaacaaggtgctgagttcattgctgagaacttgagacttcttaacgaggaagacgagctttcccctgattttcagatcattttcccagctttgcttcaaaaagctaaagcattgggtattaacttgccttacgacttgcctttcattaagtatttgtcaaccaccagagaagctcgtttaaccgacgtctccgctgcagccgataacattccagcaaacatgttgaacgcccttgagggtttggaggaagttattgactggaacaagattatgagattccagtccaaggacggttctttcctttcttctccagcctctaccgcctgcgttttgatgaacactggagatgagaagtgttttacttttctgaacaacttgttggataaatttggtggttgcgttccatgtatgtattcaatcgacctgttggagagattatcattggtggataacatcgaacacttgggaatcggtcgtcacttcaagcaagaaattaagggagctttggactatgtctacagacactggtctgagagaggtattggttggggtcgtgattctttagtccctgacctgaacaccactgctttgggtttgagaactcttagaatgcacggttacaatgtttcttctgacgttttgaacaacttcaaggatgaaaacggtagatttttctcctctgccggtcaaactcatgtcgagctgagatctgttgtcaacttgttccgtgcttctgatttggcattcccagatgaaagagctatggacgacgctagaaagtttg

SEQ ID NO: 19 - DNA, 1234 bp, Artificial Sequence
  Artificial Sequence
aagagctatggacgacgctagaaagtttgcagagccttacttgagagaagctctggccactaagatttctactaacactaaacttttcaaggagatcgagtacgttgtcgaatacccttggcacatgtctattccacgtcttgaagctagatcttacatcgattcttacgatgacaactacgtttggcagcgtaagactttatacagaatgccatcactttcaaactcaaagtgcttggaattggctaaactggacttcaacattgttcagtccttgcatcaggaggagttgaagttgttgactagatggtggaaggaatcaggtatggccgatattaacttcaccagacaccgtgttgctgaggtttacttctcctccgcaacctttgagccagagtattctgctactagaatcgctttcactaaaattggttgcttacaagtcttgttcgatgacatggctgatatcttcgctactcttgacgagcttaagtctttcactgagggagttaagcgttgggacacttccttgttacacgaaattccagaatgtatgcagacttgtttcaaagtctggttcaagttgatggaggaggttaataacgatgttgttaaggtgcaaggtagagatatgttggctcacattcgtaagccttgggagttatacttcaactgttatgttcaagagagagagtggcttgaggctggttacattccaacttttgaggaatacttgaagacttacgctatctcagtcggtttgggtccttgcactttacaacctatcctgttgatgggtgagttagtcaaggacgacgttgttgaaaaagttcactatccttctaacatgttcgaattggtgtctttgtcttggagattgactaacgacactaagacctaccaagcagagaaggctcgtggacaacaagcctctggtattgcttgttacatgaaagacaaccctggtgctaccgaggaagacgctattaagcacatttgtagagttgtcgaccgtgctcttaaggaagcatcatttgaatacttcaagccatccaacgatattccaatgggttgtaagtctttcattttcaacttaagactgtgcgttcaaattttctataagttcattgacggttacggtatcgcaaacgaagagattaaagattacattcgtaaggtctacattgacccaattcaagtctaaacgggaagtctttacagttttagttaggag

SEQ ID NO: 20 - DNA, 24 bp, Artificial Sequence
  Primer
tctaactctctttccacctgcaag

SEQ ID NO: 21 - DNA, 24 bp, Artificial Sequence
  Primer
cagctctctcctgataagtggaag

SEQ ID NO: 22 - DNA, 24 bp, Artificial Sequence
  Primer
gatgaacactggagatgagaagtg

SEQ ID NO: 23 - DNA, 59 bp, Artificial Sequence
  Primer
cttggaagtaccagtagaagaggacatttttgatgtttgatagtttgataagagtgaac

SEQ ID NO: 24 - DNA, 60 bp, Artificial Sequence
  Primer
gacttcatgtactcattgaaatcaaacattttgttcgattattctccagataaaatcaac

SEQ ID NO: 25 - DNA, 58 bp, Artificial Sequence
  Primer
cttggaagtaccagtagaagaggacattttgttcgattattctccagataaaatcaac

SEQ ID NO: 26 - DNA, 58 bp, Artificial Sequence
  Primer
gacttcatgtactcattgaaatcaaacatttttgatgtttgatagtttgataagagtg

SEQ ID NO: 27 - DNA, 57 bp, Artificial Sequence
  Primer
cttggaagtaccagtagaagaggacatttttactacgatagacacaagaagaagcag

SEQ ID NO: 28 - DNA, 59 bp, Artificial Sequence
  Primer
gacttcatgtactcattgaaatcaaacatatttattgattatttgtttatgggtgagtc

SEQ ID NO: 29 - DNA, 57 bp, Artificial Sequence
  Primer
cttggaagtaccagtagaagaggacatatttattgattatttgtttatgggtgagtc

SEQ ID NO: 30 - DNA, 59 bp, Artificial Sequence
  Primer
gacttcatgtactcattgaaatcaaacatttttactacgatagacacaagaagaagcag

SEQ ID NO: 31 - DNA, 56 bp, Artificial Sequence
  Primer
cttggaagtaccagtagaagaggacatcgtttcgaataattagttgttttttgatc

SEQ ID NO: 32 - DNA, 59 bp, Artificial Sequence
  Primer
gacttcatgtactcattgaaatcaaacattttaattgtaagtcttgactagagcaagtg

SEQ ID NO: 33 - DNA, 57 bp, Artificial Sequence
  Primer
cttggaagtaccagtagaagaggacattttaattgtaagtcttgactagagcaagtg

SEQ ID NO: 34 - DNA, 58 bp, Artificial Sequence
  Primer
gacttcatgtactcattgaaatcaaacatcgtttcgaataattagttgttttttgatc

SEQ ID NO: 35 - DNA, 55 bp, Artificial Sequence
  Primer
cttggaagtaccagtagaagaggacattgtgttttgatagttgttcaattgattg

SEQ ID NO: 36 - DNA, 57 bp, Artificial Sequence
  Primer
gacttcatgtactcattgaaatcaaacattgtgttttgatagttgttcaattgattg

SEQ ID NO: 37 - DNA, 55 bp, Artificial Sequence
  Primer
gacgaggacaccaagacatttctacaaaaataatcgaactccgaatgcggttctc

SEQ ID NO: 38 - DNA, 56 bp, Artificial Sequence
  Primer
cttggaagtaccagtagaagaggacattgttgtagttttaatatagtttgagtatg

SEQ ID NO: 39 - DNA, 60 bp, Artificial Sequence
  Primer
gacttcatgtactcattgaaatcaaacattttgatttgtttaggtaacttgaactggatg

SEQ ID NO: 40 - DNA, 2488 bp, Artificial Sequence
  Construct Das2-Das1
ttttgatgtttgatagtttgataagagtgaactttagtgtttagaggggttataatttgttgtaactggttttggtcttaagttaaaacgaacttgttatattaaacacaacggtcactcaggatacaagaataggaaagaaaaactttaaactggggacatgttgtctttatataatttggcggttaacccttaatgcccgtttccgtctcttcatgataacaaagctgcccatctatgactgaatgtggagaagtatcggaacaacccttcactaaggatatctaggctaaactcattcgcgccttagatttctccaaggtatcggttaagtttcctctttcgtactggctaacgatggtgttgctcaacaaagggatggaacggcagctaaagggagtgcatggaatgactttaattggctgagaaagtgttctatttgtccgaatttcttttttctattatctgttcgtttgggcggatctctccagtggggggtaaatggaagatttctgttcatggggtaaggaagctgaaatccttcgtttcttataggggcaagtatactaaatctcggaacattgaatggggtttactttcattggctacagaaattattaagtttgttatggggtgaagttaccagtaattttcattttttcacttcaacttttggggtatttctgtggggtagcatagcttgacaggtaatatgatgtactatgggataggcaagtcttgtgtttcagataccgccaaacgttaaataggaccctcttggtgacttgctaacttagaaagtcatgcccaggtgttacgtaatcttacttggtatgactttttgagtaacggacttgctagagtccttaccagacttccagtttagcaaaccacagattgatctgtcctctggcatatctcaaaccaatcaacacccgtaaccctttcatgaaacaactctagaatgcgtcttatcaacaggattgcccaaaacagtaattggggcggtggaatctacatgggagttccatcgttgtctcggtttttctccctataagctactctggagacgaagtaactaacaccctcaaatatcattatgtcctggtcagggttcaagaaagccgtcaatagagctggaacgcaggtccttatgaagacaaaccatcttgatgagagtctggatgaagagtttgatttccaggagaagaacttccggattatccaacaatttactcaagagctctacaatcgactttcaagcttattggaaaatcatcatagttgtctaaaggctaatctagccgttgctaccactttgaactcatattatggaacctccactacggatggatttgaaggaaaatatctggagatcgtcaacaggataaaagacgatgtgttacccaattcagtggaaccgttcaattatacaatattgcaaccgttagagactcttaaacagtacaatgaagagtttgacttgttaataaaaaaacgttatagaaagaaattggactacgatatgctccaatccaaattgtcaaaattgaccaccgaaaaagaacaattggaatttgacaagaggaacaactcactagattctcaaacggagcgtcacctagagtcagtttccaagtcaattacagaaagtttggaaacagaagaggagtatctacaattgaattccaaacttaaagtcgagctgtccgaattcatgtcgctaaggctttcttacttggaccccatttttgaaagtttcattaaagttcagtcaaaaattttcatggacatttatgacacattaaagagcggactaccttatgttgattctctatccaaagaggattatcagtccaagatcttggactctagaatagataacattctgtcgaaaatggaagcgctgaaccttcaagcttacattgatgattagagcaatgatataaacaacaattgagtgacaggtctactttgttctcaaaaggccataaccatctgtttgcatctcttatcaccacaccatcctcctcatctggccttcaattgtggggaacaactagcatcccaacaccagactaactccacccagatgaaaccagttgtcgcttaccagtcaatgaatgttgagctaacgttccttgaaactcgaatgatcccagccttgctgcgtatcatccctccgctattccgccgcttgctccaaccatgtttccgcctttttcgaacaagttcaaatacctatctttggcaggacttttcctcctgccttttttagcctcaggtctcggttagcctctaggcaaattctggtcttcatacctatatcaacttttcatcagatagcctttgggttcaaaaaagaactaaagcaggatgcctgatatataaatcccagatgatctgcttttgaaactattttcagtatcttgattcgtttacttacaaacaactattgttgattttatctggagaataatcgaacaaa

SEQ ID NO: 41 - DNA, 1818 bp, Artificial Sequence
  Construct DAS2-d8-DAS1-d2d5
ttttgatgtttgatagtttgataagagtgaactttagtgtttagaggggttataatttgttgtaactggttttggtcttaagttaaaacgaacttgttatattaaacacaacggtcactcaggatacaagaataggaaagaaaaactttaaactggggacatgttgtctttatataatttggcggttaacccttaatgcccgtttccgtctgaacaacccttcactaaggatatctaggctaaactcattcgcgccttagatttctccaaggtatcggttaagtttcctctttcgtactggctaacgatggtgttgctcaacaaagggatggaacggcagctaaagggagtgcatggaatgactttaattggctgagaaagtgttctatttgtccgaatttcttttttctattatctgttcgtttgggcggatctctccagtggggggtaaatggaagatttctgttcatggggtaaggaagctgaaatccttcgtttcttataggggcaagtatactaaatctcggaacattgaatggggtttactttcattggctacagaaattattaagtttgttatggggtgaagttaccagtaattttcattttttcacttcaacttttggggtatttctgtggggtagcatagcttgacaggtaatatgatgtactatgggataggcaagtcttgtgtttcagataccgccaaacgttaaataggaccctcttggtgacttgctaacttagaaagtcatgcccaggtgttacgtaatcttacttggtatgactttttgagtaacggacttgctagagtccttaccagacttccagtttagcaaaccacagattgatctgtcctctggcatatctcaaaccaatcaacacccgtaaccctttcatgaaacaactctagaatgcgtcttatcaacaggattgcccaaaacagtaataataaaaaaacgttatagaaagaaattggactacgatatgctccaatccaaattgtcaaaattgaccaccgaaaaagaacaattggaatttgacaagaggaacaactcactagattctcaaacggagcgtcacctagagtcagtttccaagtcaattacagaaagtttggaaacagaagaggagtatctacaattgaattccaaacttaaagtcgagctgtccgaattcatgtcgctaaggctttcttacttggaccccatttttgaaagtttcattaaagttcagtcaaaaattttcatggacatttatgacacattaaagagcggactaccttatgttgattctctatccaaagaggattatcagtccaagatcttggactctagaatagataacattctgtcgaaaatggaagcgctgaaccttcaagcttacattcctcctcatctggccttcaattgtggggaacaactagcatcccaacaccagactaactccacccagatgaaaccagttgtcgcttaccagtcaatgaatgttgagctaacgttccttgaaactcgaatgatcccagccttgctgcgtatcatccctccgctattccgccgcttgctccaaccatgtttccgcctttttcgaacatcctgccttttttagcctcaggtctcggttagcctctaggcaaattctggtcttcatacctatatcaacttttcatcagatagcctttgggttcaaaaaagaactaaagcaggatgcctgatatataaatcccagatgatctgcttttgaaactattttcagtatcttgattcgtttacttacaaacaactattgttgattttatctggagaataatcgaacaaa

SEQ ID NO: 42 - DNA, 457 bp, Artificial Sequence
  shBDP-28
ttttactacgatagacacaagaagaagcaggagggggaggatctggatatttataagagtctcccatagataacttcatgataacaaagctgcccatctatgactgaatgtggagaagtatcggaacaacccttcactaaggatatctaggctaaactcattcgcgccttagatttctccaaggtatcggttaagtttcctctttcgtactggctaacgatggtgttgctcaacaaagggatggaacgagttcaaatacctatctttggcaggacttttcctcctgccttttttagcctcaggtctcggttagcctctaggcaaattctggtcttcatacctatatcaacttttcatcagatagcctttgggttcaaaaaattaactcttttcatctataaatacaagacgagtgcgtccttttctagactcacccataaacaaataatcaataaat

SEQ ID NO: 43 - DNA, 1440 bp, Artificial Sequence
  Construct AOX1-CAT1
agtttcgaataattagttgttttttgatcttctcaagttgtcgttaaaagtcgttaaaatcaaaagcttgtcaattggaaccagtcgcaattatgaaagtaagctaataatgatgataaaaaaaaaggtttaagacagggcagcttccttctgtttatatattgctgtcaagtaggggttagaacagttaaattttgatcatgaacgttaggctatcagcagtattcccaccagaatcttggaagcatacaatgtggagacaatgcataatcatccaaaaagcgggtgtttccccatttgcgtttcggcacaggtgcaccggggttcagaagcgatagagagactgcgctaagcattaatgagattatttttgagcattcgtcaatcaataccaaacaagacaaacggtatgccgacttttggaagtttctttttgaccaactggccgttagcatttcaacgaaccaaacttagttcatcttggatgagatcacgcttttgtcatattaggttccaagacagcgtttaaactgtcagttttgggccatttggggaacatgaaactatttgaccccacactcagaaagccctcatctggagtgatgttcgggtgtaatgcggagcttgttgcattcggaaataaacaaacatgaacctcgccaggggggccaggatagacaggctaataaagtcatggtgttagtagcctaatagaaggaattggaatgagcgagctccaatcaagcccaataactgggctggtttttcgatggcaaaagtgggtgttgaggagaagaggagtggaggtcctgcgtttgcaacggtctgctgctagtgtatcccctcctgttgcgtttggcacttatgtgtgagaatggacctgtggatgtcggatggcaaaaaggtttcattcaacctttcgtctttggatgttagatcttaatcgaactccgaatgcggttctcctgtaaccttaattgtagcatagatcacttaaataaactcatggcctgacatctgtacacgttcttattggtcttttagcaatcttgaagtctttctattgttccggtcggcattacctaataaattcgaatcgagattgctagtacctgatatcatatgaagtaatcatcacatgcaagttccatgataccctctactaatggaattgaacaaagtttaagcttctcgcacgagaccgaatccatactatgcacccctcaaagttgggattagtcaggaaagctgagcaattaacttccctcgattggcctggacttttcgcttagcctgccgcaatcggtaagtttcattatcccagcggggtgatagcctctgttgctcatcaggccaaaatcatatataagctgtagacccagcacttcaattacttgaaattcaccataacacttgctctagtcaagacttacaattaaa

SEQ ID NO: 44 - DNA, 1426 bp, Artificial Sequence
  Construct AOX1-GAP
cgtttcgaataattagttgttttttgatcttctcaagttgtcgttaaaagtcgttaaaatcaaaagcttgtcaattggaaccagtcgcaattatgaaagtaagctaataatgatgataaaaaaaaaggtttaagacagggcagcttccttctgtttatatattgctgtcaagtaggggttagaacagttaaattttgatcatgaacgttaggctatcagcagtattcccaccagaatcttggaagcatacaatgtggagacaatgcataatcatccaaaaagcgggtgtttccccatttgcgtttcggcacaggtgcaccggggttcagaagcgatagagagactgcgctaagcattaatgagattatttttgagcattcgtcaatcaataccaaacaagacaaacggtatgccgacttttggaagtttctttttgaccaactggccgttagcatttcaacgaaccaaacttagttcatcttggatgagatcacgcttttgtcatattaggttccaagacagcgtttaaactgtcagttttgggccatttggggaacatgaaactatttgaccccacactcagaaagccctcatctggagtgatgttcgggtgtaatgcggagcttgttgcattcggaaataaacaaacatgaacctcgccaggggggccaggatagacaggctaataaagtcatggtgttagtagcctaatagaaggaattggaatgagcgagctccaatcaagcccaataactgggctggtttttcgatggcaaaagtgggtgttgaggagaagaggagtggaggtcctgcgtttgcaacggtctgctgctagtgtatcccctcctgttgcgtttggcacttatgtgtgagaatggacctgtggatgtcggatggcaaaaaggtttcattcaacctttcgtctttggatgttagatcttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 45 - DNA, 986 bp, Artificial Sequence
  Construct GAP-CAT1
tgtgttttgatagttgttcaattgattgaaatagggacaaataaattaaatttaaagtctttgggtcaggagaaaccaaaattgggaaaggtgttcgccttttatattcgattctggtggtttccaataatctcatgacatgcgtccgcccgctattattgccagcgacggccgggacttttccatccctgggctgctaggtcgggtacacgacctccgttttacccgcaacgtaatgctgggaagagcattgctgcaagggggccgtagaagaagctctccagcagagtaaaatttcctagggacggtaacgggcggtggaaggagagagaagggaagagacgtttctggttccattactccacatttaagttttaccccggagaattttacgttgccagcaggtcgttcggagttgcaacggagccagatatttcagagatggctacctgattggacgaggacaccaagacatttctacaaaaataatcgaactccgaatgcggttctcctgtaaccttaattgtagcatagatcacttaaataaactcatggcctgacatctgtacacgttcttattggtcttttagcaatcttgaagtctttctattgttccggtcggcattacctaataaattcgaatcgagattgctagtacctgatatcatatgaagtaatcatcacatgcaagttccatgataccctctactaatggaattgaacaaagtttaagcttctcgcacgagaccgaatccatactatgcacccctcaaagttgggattagtcaggaaagctgagcaattaacttccctcgattggcctggacttttcgcttagcctgccgcaatcggtaagtttcattatcccagcggggtgatagcctctgttgctcatcaggccaaaatcatatataagctgtagacccagcacttcaattacttgaaattcaccataacacttgctctagtcaagacttacaattaaa

SEQ ID NO: 46 - DNA, 550 bp, Artificial Sequence
  Construct HTA1-HTB1
tgttgtagttttaatatagtttgagtatgagatggaactcagaacgaaggaattatcaccagtttatatattctgaggaaagggtgtgtcctaaattggacagtcacgatggcaataaacgctcagccaatcagaatgcaggagccataaattgttgtattattgctgcaagatttatgtgggttcacattccactgaatggttttcactgtagaattggtgtcctagttgttatgtttcgagatgttttcaagaaaaactaaaatgcacaaactgaccaataatgtgccgtcgcgcttggtacaaacgtcaggattgccaccacttttttcgcactctggtacaaaagttcgcacttcccactcgtatgtaacgaaaaacagagcagtctatccagaacgagacaaattagcgcgtactgtcccattccataaggtatcataggaaacgagagtcctccccccatcacgtatatataaacacactgatatcccacatccgcttgtcaccaaactaatacatccagttcaagttacctaaacaaatcaaa

SEQ ID NO: 47 - DNA, 365 bp, Artificial Sequence
  Construct HHT2-HHF2
ttttactacgatagacacaagaagaagcaggagggggaggatctggatatttataagagtctcccatagataacgatttggcactttttgccatcagtgccaacagtatttcgcactgcgacactcccgactgaaatgggatgcaagtttattatgagttctggtagcatagaaatgggacatgttcttacagtttcaaatttacgcacgctctgcctctaggagtacggctcagttcatcgcgtaccgtgtcgtatcaacattacggtttggcactgcattgtccaccttaactcttttcatctataaatacaagacgagtgcgtccttttctagactcacccataaacaaataatcaataaat